(19) United States
(12) Patent Application Publication
(10) Pub. No.: US 2018/0122517 A1
(43) Pub. Date: May 3, 2018

(54) METHODS AND APPARATUS RELATED TO ELECTRONIC DISPLAY OF A HUMAN AVATAR WITH DISPLAY PROPERTIES PARTICULARIZED TO HEALTH RISKS OF A PATIENT

(71) Applicant: Patient Identification Platform, Inc., Victor, NY (US)

(72) Inventor: Russell Bessette, Louisville, KY (US)

(21) Appl. No.: 15/561,865

(22) PCT Filed: Mar. 24, 2016
(86) PCT No.: PCT/US2016/024007

§ 371 Date: Sep. 26, 2017

Related U.S. Application Data
(60) Provisional application No. 62/139,345, filed on Mar. 27, 2015.

(51) Int. Cl. G16H 50/30; G16H 20/30; G16H 50/50

(52) U.S. Cl. CPC G16H 50/30; G16H 40/67; G16H 50/50; G16H 20/30

(57) ABSTRACT

Methods, apparatus, and computer storage media related to generating an electronic display of a human avatar with display properties that are particularized to health risks of a patient. The display properties may include display properties for graphical representations of human organs to be presented in the electronic display in combination with the human avatar. Each of the display properties of the graphical representations of human organs may be based on a magnitude of an organ health risk score that is calculated for a corresponding organ based on test values (for one or more selected medical test results for the patient) that are generally indicative of function of the organ. In some implementations, the display properties may also include an indication of an overall health risk score that is based on a plurality of the test values for the patient.

### BACKGROUND

Healthcare costs represent a substantial portion of the gross national product of many countries such as the United States. In view of the costs associated with healthcare, consumers and providers are driven to seek lower costs yet retain quality. However, patient specific impacts of a given illness are difficult to determine because the same illness may impact people differently due to, for example, personal physiology, treatment choice, provider skills/knowledge, and patient compliance with treatments or other therapies. Moreover, patients and/or other parties may have difficulty fully understanding the impacts of various illnesses and/or the impacts of various treatments or other therapies on those illnesses.

These and/or other factors may contribute to the stifling of good public health, increased healthcare costs, poor patient/provider communications, and/or other issues. As one example, a patient's lack of understanding of the impacts of a given medical diagnosis and lack of understanding of the impact of therapy on the given medical diagnosis may cause the patient to not take the given medical diagnosis and therapy seriously—thereby contributing to negative impacts on the patient's health (and the public health in general), and eventual increased healthcare costs for the patient.

### SUMMARY

This specification is directed generally to generating an electronic display with display properties that are particularized to health risks for a patient based on that patient's personal medical test results, and which produce a probability score predicting the need for increasing levels of medical care. The display properties may include display properties for graphical representations of human organs to be presented in the electronic display in combination with a human avatar. Each of the display properties for the graphical representations of human organs may be based on a magnitude of an organ health risk score that is calculated for a corresponding organ based on test values (for one or more selected medical test results for the patient) that are generally indicative of function of the organ. For example, a display property of a graphical representation of a pancreas may be a color of “red” based on an organ health risk score for the pancreas indicating a large degree of dysfunction of the organ; whereas a display property of a graphical representation of a heart may be a color of “yellow” based on a health risk score for the heart indicating a mild degree of dysfunction of the organ. In some implementations, the display properties may also include an indication of an overall health risk score that is based on a plurality of the test values for the patient.

In some implementations, the overall health risk score and/or individual organ health risk scores for a patient having a medical diagnosis may be calculated based on applying regression correlation coefficients to test values of the patient for one or more selected medical test results of the patient. Each regression correlation coefficient may indicate a statistically calculated historical impact of test values for one of the medical results on a medical condition indicated by the medical diagnosis, such as a statistically calculated historical impact on increasing levels of medical care required to treat increasing levels of medical illness. In some implementations, the overall health risk score may be calculated based on an illness severity component, an illness volatility component, an illness complexity component, and/or a disease progression component.

In some implementations, an overall health risk score and/or individual organ health risk scores may be calculated for each of a plurality of time periods and the display properties of the electronic display may be modified to illustrate changes to the health risk score and/or individual organ health risk scores over the time periods. For example, a time period adjustment input may be received to switch between multiple time periods and the display properties of the electronic display updated based on the appropriate health risk score and/or individual organ health risk scores. For instance, the electronic display may be provided with display properties based on a health risk score and individual organ health risk scores for a “current” time period then, in response to a time period adjustment input, the display properties may be updated based on a health risk score and individual organ health risk scores for a “past” or “future” time period switched to as indicated by the time period adjustment input. Health risk scores and/or individual organ health risk scores of a patient that are calculated for a “future” time period may be based on, for example, therapy input that indicates actions performed or performable by the patient such as biometric data that indicates actual or anticipated: heart rate, dietary values, body mass index (“BMI”), activity values, and/or sleep values of the patient.

In some implementations, the generated electronic display enables improved understanding of: a patient's medical diagnosis, potential risks to the patient's overall health, potential risks to specific organs of the patients, and/or potential changes to the patient's overall and/or organ health that may result with conformance to particular therapy. In some of those implementations, the electronic display may increase the likelihood of the patient participating in appropriate therapy and/or other treatment options to address the patient's medical diagnosis, thereby promoting good public health.

Generally, in one aspect, a computer implemented method is provided that comprises: identifying, from one or more electronic databases, patient data for a patient identifier, the patient data comprising test values based on results for one or more selected medical tests for a patient associated with the patient identifier; calculating, utilizing one or more processors, an overall health risk score for the patient identifier based on the test values; calculating, utilizing one or more of the processors, organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ; determining display properties for graphical representations of the organs based on the organ health risk scores, wherein each of the display properties is determined for a corresponding of the organs, and wherein a given display property for the given organ is determined based on a magnitude of the given organ health risk score; and generating an electronic display that includes an avatar for the patient, the graphical representations of the organs with the display properties, and an indication of the overall health risk score.

These and/or other implementations of the technology disclosed herein may optionally include one or more of the following features.

In some implementations, the electronic display includes the graphical representations of the organs with the display properties in anatomically appropriate positions in the avatar.

In some implementations, the test values include a first group of test values associated with a first time period and a second group of test values associated with a second time period, and the overall health risk score and the individual organ health risk scores are calculated based on the first group of test values. In some of those implementations, the method further comprises: calculating, utilizing one or more of the processors, a second overall health risk score for the patient identifier based on the second group of test values; calculating, utilizing one or more of the processors, second organ health risk scores for the patient identifier based on the second group of test values, wherein a given second organ health risk score of the second organ health risk scores for the given organ is calculated based on one or more of the second group of test values that are indicative of function of the given organ; determining second display properties for the graphical representations of the organs based on the second organ health risk scores; and modifying the electronic display to display the graphical representations of the organs with the second display properties, the second overall health risk score, and an indication of the second time period. In some of those implementations, the method further comprises receiving a time period adjustment input—and modifying the electronic display to display the graphical representations of the organs with the second display properties, the second overall health risk score, and the indication of the second time period is in response to receiving the time period adjustment input. The time period adjustment input may be received responsive to user interaction with an adjustable user interface element of the electronic display and the indication of the second time period may be based on a current state of the adjustable user interface element.

In some implementations, the method further comprises: determining additional display properties associated with additional graphical representations of multiple of the organs, wherein each of the additional display properties is determined based on the organ health risk scores and is determined for a corresponding of the organs, and wherein a given additional display property associated with the given organ is determined based on a magnitude of the given organ health risk score and provides more detailed information than the given display property for the given organ; wherein generating the electronic display further includes generating the additional graphical representations of the multiple of the organs along with the additional display properties, the additional graphical representations and the additional display properties depicted exterior of the avatar in the electronic display. In some of those implementations, the method further comprises ordering the additional graphical representations of the multiple of the organs in the electronic display based on the individual organ health risk scores. The given additional display property may comprise a numerical indication of the given organ health risk score and/or a bar graph indicating a magnitude of the organ health risk score.

In some implementations, the display properties include a plurality of colors each mapped to one or more of the individual organ health risk scores.

In some implementations, the method further comprises: identifying therapy input indicative of actions performed or performable by the patient; calculating, utilizing one or more of the processors, a modified overall health risk score for the patient identifier based on the test values and the therapy input; and modifying the electronic display to display the modified overall health risk score. In some of those implementations, the therapy input is received from a personal fitness monitoring device of the patient and indicates actions performed by the patient. In other of those implementations, the therapy input includes actions performable by the patient to improve the overall health risk score and the modified overall health risk score indicates an anticipated potential future health risk score if the actions performable by the patient are actually performed by the patient. In yet other of those implementations, the method further comprises: calculating, utilizing one or more of the processors, anticipated future organ health risk scores for the patient identifier based on the test values and the therapy input; determining second display properties for the graphical representations of the organs based on the anticipated future organ health risk scores; and modifying the electronic display to display the graphical representations of the organs with the second display properties along with the modified overall health risk score.

In some implementations, calculating the overall health risk score for the patient identifier based on the test values comprises: identifying coefficient values for each of the test values, the coefficient values indicating a statistically calculated historical impact, for a medical diagnosis of the patient, of the test values on predicting an increased need for medical care and associated costs; and modifying each of the test values in view of a respective of the coefficient values. The test values may be z-scores.

In some implementations, calculating the overall health risk score for the patient identifier based on the test values comprises one or more of: calculating an illness severity component of the overall health risk score based on a ratio of Blood Urea Nitrogen levels and serum Creatinine levels of the patient as determined based on one or more of the test values; and calculating an illness volatility component of the overall health risk score based on variations over time for the Blood Urea Nitrogen levels of the patient and variations over time for the serum Creatinine levels of the patient, as determined based on one or more of the test values. In some of those implementations, the method further comprises: determining an illness severity graphical indicator based on the magnitude of the illness severity component of the overall health risk score; determining an illness complexity graphical indicator based on the magnitude of the illness complexity component of the overall health risk score; and including the illness severity graphical indicator and the illness complexity graphical indicator in the electronic display. Including the illness severity graphical indicator and the illness complexity graphical indicator in the electronic display may comprise modifying the display in response to receiving a selection for additional information related to the overall health risk score.

In some implementations, calculating the overall health risk score for the patient identifier based on the test values comprises one or more of: calculating a disease stage/progression component of the overall health risk score based on disease progression data of the patient data, the disease progression data indicating an extent of a medical diagnosis of the patient; and calculating an illness complexity component of the overall health risk score, the illness complexity component based on one or more selected test values of the test values, the selected test values excluding medical test results that define the medical diagnosis of the patient.

In some implementations, the selected medical tests include at least one physical measurement medical test and at least one laboratory measurement medical test.

In some implementations, the test values include values based on one or more of: complete blood counts with white cell differential, serum electrolytes, liver profile enzymes, metabolic study values, estimated glomerular filtration rate, urine analysis, selected tumor markers, genetic markers, thyroid, lipid and tri-glyceride values, total cholesterol and ratio high-density lipoprotein (HDL) & low-density lipoprotein (LDL), blood pressure, body mass index, waist circumference, and/or patient health questionnaire-9 (PHQ-9) results.

Generally, in another aspect, a computer implemented method is provided that comprises: identifying, from one or more electronic databases, patient data for a patient identifier, the patient data comprising test values based on results for one or more selected medical tests for a patient associated with the patient identifier; calculating, utilizing one or more processors, an overall health risk score for the patient identifier based on the test values; calculating, utilizing one or more of the processors, organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ; generating an electronic display that includes an indication of the overall health risk score and indications of the organ health risk scores; identifying therapy input indicative of actions performed or performable by the patient; calculating, utilizing one or more of the processors, a second overall health risk score for the patient identifier based on the test values and the therapy input; calculating, utilizing one or more of the processors, second organ health risk scores for the patient identifier based on the test values and the therapy input; and generating a modified electronic display that includes an indication of the second overall health risk score and indications of the second organ health risk scores.

These and/or other implementations of the technology disclosed herein may optionally include one or more of the following features.

In some implementations, the therapy input is received from a personal fitness monitoring device of the patient and indicates actions performed by the patient.

In some implementations, the method further comprises determining display properties for graphical representations of the organs based on the organ health risk scores. Each of the display properties is determined for a corresponding of the organs, a given display property for the given organ is determined based on a magnitude of the given organ health risk score, and the indications of the organ health risk scores in the electronic display comprise the graphical representations of the organs with the display properties. In some of those implementations, the method further comprises determining second display properties for the graphical representations of the organs based on the second organ health risk scores. Each of the second display properties is determined for a corresponding of the organs and the indications of the organ health risk scores in the modified electronic display comprise the graphical representations of the organs with the second display properties. The graphical representations of the organs in the electronic display may be provided in anatomically appropriate positions in an avatar for the patient and the graphical representations of the organs in the modified electronic display may be provided in the anatomically appropriate positions in the avatar for the patient. The display properties may include a first set of colors mapped to the organ health risk scores and the second display properties may include a second set of colors mapped to the second organ health risk scores. In some implementations, the method further comprises: determining additional display properties associated with additional graphical representations of multiple of the organs, wherein each of the additional display properties is determined based on the organ health risk scores and is determined for a corresponding of the organs, and wherein a given additional display property associated with the given organ is determined based on a magnitude of the given organ health risk score and provides more detailed information than the given display property for the given organ; wherein generating the electronic display further includes generating the additional graphical representations of the multiple of the organs along with the additional display properties, the additional graphical representations and the additional display properties depicted exterior of the avatar in the electronic display; determining second additional display properties associated with second additional graphical representations of multiple of the organs, wherein each of the second additional display properties is determined based on the second organ health risk scores and is determined for a corresponding of the organs; wherein generating the modified electronic display further includes generating the second additional graphical representations of the multiple of the organs along with the additional second display properties, the additional second graphical representations and the additional second display properties depicted exterior of the avatar in the modified electronic display.

In some implementations, calculating the second overall health risk score for the patient identifier based on the test values and the therapy input comprises: identifying, based on the therapy input, a predicted change for each of one or more affected test values of the test values; modifying each of the affected test values to create one or more modified test values, the modifying of a given affected test value of the affected test values comprising modifying the given affected test value in view of the predicted change for the given affected test value; and calculating the modified overall health risk score based on the one or more modified test values.

In some implementations, calculating the second organ health risk scores for the patient identifier based on the test values and the therapy input comprises: identifying, based on the therapy input, a predicted change to a given test value of the one or more test values that are indicative of function of the given organ; modifying the given test value based on the predicted change to create a modified given test value; and calculating, for the given organ, a second organ health risk score of the second organ health risk scores based on the modified given test value. In some of those implementations, calculating the second organ health risk score based on the modified given test value comprises: identifying a coefficient value for the modified given test value, the coefficient value indicating a statistically calculated historical impact, for a medical diagnosis of the patient, of the modified given test value on predicting an increased need for medical care and associated costs; and modifying the modified given test value in view of the coefficient value.

In some implementations, the therapy input includes actions performable by the patient to improve the overall health risk score and the second overall health risk score indicates an anticipated potential future health risk score if the actions performable by the patient are actually performed by the patient. In some of those implementations, the therapy input is received via user interaction with the electronic display.

In some implementations, calculating the overall health risk score for the patient identifier based on the test values comprises one or more of: calculating an illness severity component of the overall health risk score based on a ratio of Blood Urea Nitrogen levels and serum Creatinine levels of the patient as determined based on one or more of the test values; and calculating an illness volatility component of the overall health risk score based on variations over time for the Blood Urea Nitrogen levels of the patient and variations over time for the serum Creatinine levels of the patient, as determined based on one or more of the test values. In some of those implementations, the method further comprises: determining an illness severity graphical indicator based on the magnitude of the illness severity component of the overall health risk score; determining an illness complexity graphical indicator based on the magnitude of the illness complexity component of the overall health risk score; and including the illness severity graphical indicator and the illness complexity graphical indicator in the electronic display.

Other implementations may include one or more non-transitory computer readable storage media storing instructions executable by a processor to perform a method such as one or more of the methods described above. Yet another implementation may include a system including memory and one or more processors operable to execute instructions, stored in the memory, to perform a method such as one or more of the methods described above. Yet another implementation may include a system that accepts inputs from one or more passive bio-metric sensors that trigger computations which impact predicted overall health risk scores and/or organ health risk scores.

It should be appreciated that all combinations of the foregoing concepts and additional concepts described in greater detail herein are contemplated as being part of the subject matter disclosed herein. For example, all combinations of claimed subject matter appearing at the end of this disclosure are contemplated as being part of the subject matter disclosed herein.

### BRIEF DESCRIPTION OF THE DRAWINGS

FIG. 1 illustrates an example environment in which an electronic display of a human avatar with display properties that are particularized to health risks of a patient may be generated.

FIG. 2 illustrates an example of calculating an overall health risk score and organ health risk scores based on patient data, generating an electronic display with display properties determined based on the calculated scores, and adjusting the electronic display based on user input and/or therapy input.

FIG. 3A illustrates an example of an electronic display of an avatar for a patient with an indication of an overall health risk score for the patient at a first time period and graphical representations of organs with display properties of the organs determined based on organ health risk scores for the organs at the first time period.

FIG. 3B illustrates the example electronic display of FIG. 3A, with the display modified to provide an indication of an overall health risk score for the patient at a second time period and graphical representations of organs with display properties of the organs determined based on organ health risk scores for the organs at the second time period.

FIG. 3C illustrates the example electronic display of FIG. 3A, modified in response to a user selection of graphical representations of two organs.

FIG. 4A illustrates another example of an electronic display of an avatar for a patient with an indication of biometric data therapy input, an indication of an overall health risk score for the patient, and graphical representations of organs with display properties of the organs determined based on organ health risk scores for the organs.

FIG. 4B illustrates the example electronic display of FIG. 4A, with the display modified based on anticipated biometric data therapy input and a future time period adjustment to provide an indication of an overall health risk score for the patient that is calculated based on the anticipated biometric data therapy input and the future time period and graphical representations of organs with display properties of the organs determined based on organ health risk scores for the organs that are calculated based on the anticipated biometric data therapy input and the future time period.

FIG. 4C illustrates the example electronic display of FIG. 4B, with the display modified to provide an “illness map” that refines and further specifies the overall health risk score of FIG. 4B.

FIG. 5 is a flow chart illustrating an example method of calculating an overall health risk score and organ health risk scores based on patient data and generating an electronic display with display properties determined based on the calculated scores.

FIG. 6 is a flow chart illustrating an example method of calculating a regression correlation coefficient for a medical test result for a medical diagnosis.

FIG. 7 illustrates an example architecture of a computer system.

### DETAILED DESCRIPTION

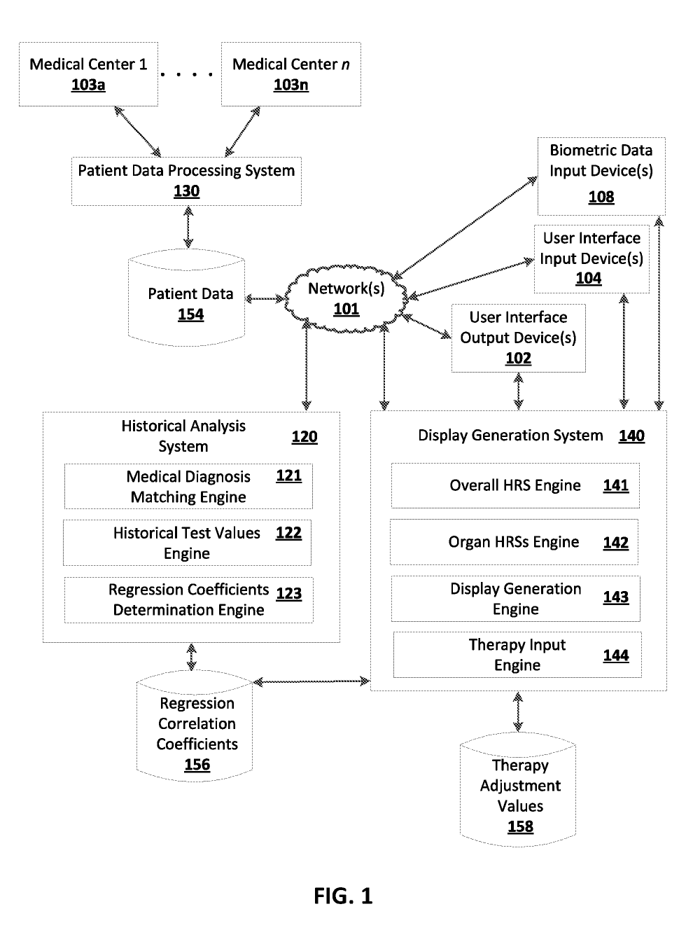

FIG. 1 illustrates an example environment in which an electronic display of a human avatar with display properties that are particularized to health risks of a patient may be generated. The example environment of FIG. 1 includes a historical analysis system 120, a patient data processing system 130, a display generation system 140, medical center systems 103a-n, one or more user interface output devices 102, one or more user interface input devices 104, and one or more biometric data input devices 108. The example environment further includes a patient data database 154, a regression correlation coefficients database 156, a therapy adjustment values database 158, and one or more networks 101 that facilitate communication between the components of the environment. The networks 101 may include, for example, a local area network (LAN) and/or wide area network (WAN) (e.g., the Internet).

The historical analysis system 120, the patient data processing system 130, the display generation system 140, and/or other components of the example environment may be implemented in one or more computers that communicate, for example, through one or more networks. The display generation system 140 is an example system in which the systems, components, and techniques described herein may be implemented and/or with which systems, components, and techniques described herein may interface. One or more components of the display generation system 140 and/or the historical analysis system 120 may be incorporated in a single system in some implementations. Also, in some implementations one or more components of the display generation system 140 may be incorporated on a device that includes one or more of the user interface output devices 102 and/or one or more of the user interface output devices 104. For example, all or aspects of display generation engine 143 may be incorporated on a client computing device, such as a tablet computing device that includes one of the user interface output devices 102 (the display component of a touch/display screen of the tablet computing device) and one of the user interface input devices 104 (the touch sensitive component of the touch/display screen of the tablet computing device). Another example of a client device on which all or aspects of display generation engine 143 may be implemented is a wearable computing device such as wearable glasses that include one of the user interface output devices and/or one of the user interface input devices 104.

Generally, the patient data processing system 130 collects electronic patient data from a plurality of medical center systems 103a-n and/or other sources, optionally normalizes and/or otherwise alters one or more aspects of the patient data, and stores the optionally altered patient data in a patient data database 154. The patient data processing system 130 may collect electronic patient data from various sources such as medical center systems 103a-n that may be associated with hospitals, doctor offices, universities, personal physical fitness databases, state-wide regional health organizations, HIES (Health Information Exchanges), laboratory testing facilities, and/or other health facilities. In some implementations, the patient data processing system 130 may receive patient data via one or more standard transfer and/or data protocols such as Health Level-7 (HL7) or Admit Discharge Transfer (ADT). Patient data received by patient data processing system 130 may include, for each a plurality of patients, an optionally anonymized patient identifier, demographic information, health provider(s) information, information for one or more patient diagnoses, medical test results information, medical costs information, and/or other data. Additional description of some of the data that may be included in patient data for one or more patients is provided herein.

In some implementations, the patient data processing system 130 stores collected patient data in patient data database 154 and assigns a unique patient identifier to each patient's patient data. In some of those implementations, the patient data processing system 130 may generate a unique patient identifier for patient data of a patient based on one or more of a date associated with the patient data of the patient (e.g., date of initial collection of the patient data by patient data processing system 130, date of initial diagnosis), a value associated with a doctor or other health professional indicated by the patient data of the patient, and/or a value associated with the medical center system(s) 103a-n that provided the patient data. The patient data processing system 130 may also encrypt the patient data of patient data database 154. For example, the patient data processing system 130 may assign a cryptographic key to a patient's patient data, and provide the patient identifier and electronically provide the key to the medical center system(s) 103a-n that provided the patient data and/or to the patient. In some implementations, the patient data processing system 130 may validate the identity of each patient for which patient data is collected, and determine whether that patient already exists in the patient data database 154 prior to creating a new entry in the patient data database 154.

In this specification, the term “database” will be used broadly to refer to any electronic collection of data. The data of the database does not need to be structured in any particular way, or structured at all, and it can be stored on storage devices in one or more locations. Thus, for example, the patient data database 154 may include multiple collections of data, each of which may be organized and accessed differently. Also, in this specification, the term “entry” will be used broadly to refer to any mapping of a plurality of associated information items. A single entry need not be present in a single storage device and may include pointers or other indications of information items that may be present in unique segments of a storage device and/or on other storage devices. For example, an entry that identifies a patient identifier and patient data for the patient identifier in patient data database 154 may include multiple nodes mapped to one another, with one or more nodes including a pointer to another information item that may be present in another data structure and/or another storage medium.

In some implementations, the patient data processing system 130 may normalize one or more values associated with medical test results information, cost information, and/or other information of the patient data. Normalization of test values associated with medical test results information may, inter alio, ameliorate problems associated with different laboratories utilizing different test equipment and/or having different calibrations of test equipment. For example, for a first laboratory, “normal” test values for a particular medical test result may be from 100 to 120, whereas for a second laboratory, “normal” test values for that particular medical test result may be from 108 to 125. Normalization of values associated with cost information may, inter alio, ameliorate problems associated with regional and/or inflationary, insurance deductible requirements or other cost variations. In some implementations, the patient data processing system 130 may normalize test values associated with a medical test result by calculating a z-score for each of those values. In some of those implementations, the z-score for a patient's test value for a medical test result may be calculated based on a formula such as:

z-score=(patient's value for medical test result)−(mean of the value for the medical test result for a population of patients/standard deviation of the value for the medical test result for the population of patients).

In some implementations, the patient data processing system 130 may not normalize test values for one or more medical test results. In some of those implementations, historical analysis system 120 and/or the display generation system 140 may optionally normalize values for one or more of those medical test results. Also, it is noted that some medical test results may be associated with values that will not be normalized, such as values that identify presence or absence of specific cell markers and/or gene sequences for a patient that have been identified utilizing one or more medical tests.

In some implementations, the test values included in patient data database 154 that are used by the historical analysis system 120 and/or the display generation system 140 include, but are not limited to, values based on medical test results that indicate one or more of: complete blood counts with white cell differential, serum electrolytes, liver profile enzymes, metabolic study values, estimated glomerular filtration rate, BUN (blood urea nitrogen) to Creatinine ratio, selected tumor markers, NCI (National Cancer Institute) identified genetic tumor markers, lipid and tri-glyceride values, total cholesterol and ratio high-density lipoprotein (HDL) & low-density lipoprotein (LDL), Urine analysis for glucose, albumin and cellular proteins, blood pressure, body mass index, waist circumference, and/or patient health questionnaire-9 (PHQ-9) results.

In some implementations, the patient data processing system 130 may index the patient data of patient data database 154 to enable more efficient searching of the patient data in identifying patient identifiers that match medical diagnosis criteria and/or other criteria. For example, patient data database 154 may include a plurality of entries, with each entry being associated with a patient identifier and including patient data for that patient identifier. The patient data processing system 130 may generate an index of the entries based on one or more properties of the entries. For instance, an index may include one or more values associated with each entry, wherein the values each indicate at least one medical diagnosis associated with a respective entry.

Generally, the historical analysis system 120 calculates, for each of a plurality of medical diagnoses, regression correlation coefficients for a plurality of medical test results. Each regression correlation coefficient for a medical diagnosis indicates a statistically calculated historical impact of a test value of a medical test result on patient health for patients having the medical diagnosis. In some implementations, the historical impact of a value of a medical test result on patient health may be a historical impact on medical costs, such as a historical impact of the value on medical costs being incurred that satisfy a “high cost” threshold. A “high cost” threshold may be defined based on various factors such as, for example, average or other statistical measures of health care costs compiled by one or more entities such as FAIR HEALTH, INC. In some implementations, the “high cost” threshold for a medical diagnosis may be the same threshold as that used for other (e.g., all other) medical diagnoses. In some implementations, the “high cost” threshold may be particularized to the medical diagnosis for which regression correlation coefficients are being calculated by the historical analysis system 120. For example, the “high cost” threshold for a “chronic kidneys disease” diagnosis may be different than the “high cost” threshold for a “prostate cancer” diagnosis.

As described herein, the calculated regression correlation coefficients for a medical diagnosis may be utilized by the display generation system 140 to calculate overall health risk scores for patients having the medical diagnosis. For example, where each of the regression correlation coefficients for a medical diagnosis indicate a statistically calculated historical impact of a test value of a medical test result on medical costs, the display generation system 140 may utilize the regression correlation coefficients and test values for patients to calculate overall health risk scores for the patients that each indicate a probability that a respective patient will incur increased medical costs, such as medical costs that satisfy a high cost threshold.

As also described herein, one or more regression correlation coefficients calculated by the historical analysis system 120 may each be assigned to one or more organs whose health is indicated by the medical test result associated with the regression correlation coefficient, thereby indicating a statistically calculated historical impact on health of the patient as a result of the health of that organ. For example, the display generation system 140 may utilize one or more regression correlation coefficients associated with medical test results that indicate the health of a liver and the test values for the liver for a patient, to calculate the impact of health of the liver of the patient on increased medical costs.

In various implementations historical analysis system 120 may include a medical diagnosis matching engine 121, a historical test values engine 122, and/or a regression coefficients determination engine 123. In some implementations, all or aspects of engines 121, 122, and/or 123 may be omitted. In some implementations, all or aspects of engines 121, 122, and/or 123 may be combined. In some implementations, all or aspects of engines 121, 122, and/or 123 may be implemented in a component that is separate from historical analysis system 120.

Generally, medical diagnosis matching engine 121 identifies a medical diagnosis, and optionally one or more additional mandatory matching criteria, for which regression coefficients are to be calculated. The medical diagnosis matching engine 121 identifies a set of patient identifiers from patient data database 154 that have the medical diagnosis and the optional one or more additional mandatory matching criteria. The medical diagnosis may be, for example, “cancer”, “colon cancer”, “thyroid cancer”, “chronic kidneys disease” (CKD), or “diabetes”. In some implementations, the medical diagnosis may be defined based on an International Classification of Diseases (ICD) code and/or other accepted standard(s) and the patient data of patient data database 154 may also define diagnoses based on the ICD code and/or other accepted standard(s).

In some implementations, the optional additional mandatory matching criteria may identify an extent of the medical diagnosis such as one or more specific stages of a medical diagnosis of cancer (e.g., a stage defined by tumor size, lymph node involvement, and/or metastasis). As another example, a medical diagnosis may be, for example, “CKD”, and additional mandatory matching criteria may further define a specific stage of the CKD such as a subset of stages 0 to V. In some implementations, the optional additional mandatory matching criteria may additionally or alternatively include one or more criteria that are not directly tied to the medical diagnosis. For example, other optional additional mandatory matching criteria may include, for example, gender criteria, age criteria (e.g., a particular age range), additional diagnosis criteria (e.g., an additional diagnosis that is distinct from the primary diagnosis), etc. The additional mandatory matching criteria may be utilized, for example, to identify a particular demographic group and/or disease stage group for which regression correlation coefficients are calculated. In some implementations, one or more criteria that may be used as additional mandatory matching criteria for a medical diagnosis may instead be used as test values in determining, for the medical diagnosis, a regression correlation coefficient for a medical test result.

Generally, the historical test values engine 122 compiles sets of test values and cost indications from the patient data database 154 for the set of patient identifiers identified by medical diagnosis matching engine 121. Each set of test values and cost indications is for one of the identified patient identifiers and includes one or more test values that substantially correspond in time with any other test values for the set (e.g., all test values for a set are from medical tests conducted on the same day, within a week of one another, or within another threshold time period of one another) and that optionally substantially correspond in time with the medical diagnosis for the patient identifier (e.g., one or more test values of the set result from medical test(s) conducted while the patient had the medical diagnosis, or within a threshold time of having the medical diagnosis). Moreover, each set of test values and cost indications includes a cost indication that indicates whether high cost medical care was incurred by the patient associated with the patient identifier within a threshold time period associated with the one or more test values of the set (e.g., within two months, within one month, or within two weeks of the date of the medical tests that resulted in the test values).

As one example, for a first patient identifier identified by the medical diagnosis matching engine 121, the historical test values engine 122 may identify, from patient data database 154, one or more test values that resulted from one or more medical tests performed on 1/1/15 and a cost indication that indicates less than a high cost threshold for medical care was incurred for the patient identifier within one month of 1/1/15. For instance, the historical test values engine 122 may determine the cost indication based on calculating a sum of all medical costs that are indicated for the patient identifier in the patient data database 154 and that are associated with a time stamp from 1/1/15 to 2/1/15, and determining the sum is less than a high cost threshold. The historical test values engine 122 may further identify, from the patient data database 154, one or more second test values for the patient identifier that resulted from another iteration, performed on 2/15/15, of the one or more medical tests and a cost indication that indicates more than a high cost threshold for medical care was incurred for the patient identifier within one month of 2/15/15. For instance, the historical test values engine 122 may determine the cost indication based on calculating a sum of all medical costs that are indicated for the patient identifier in the patient data database 154 and that are associated with a time stamp from 2/15/15 to 3/15/15, and determining the sum is greater than or equal to a high cost threshold. The historical test values engine 122 may optionally identify additional sets of test values and cost indications for the first patient identifier.

For a second patient identifier identified by the medical diagnosis matching engine 121, the historical test values engine 122 may identify, from patient data database 154, one or more first test values that resulted from the one or more first medical tests being performed for the second patient identifier on 11/1/14 and a cost indication that indicates less than a high cost threshold for medical care was incurred for the second patient identifier within one month of 11/1/14. The historical test values engine 122 may optionally identify additional sets of test values and cost indications for the second patient identifier. The historical test values engine 122 may continue to identify sets of test values and cost indications for additional patient identifiers identified by the medical diagnosis matching engine 121 until all identified patient identifiers have been addressed, a threshold number of identified patient identifiers have been addressed, and/or other criterion has been satisfied. In some implementations, the test values and/or medical costs may be normalized by the historical test values engine 122 and/or may already be normalized in the patient data database 154.

In some implementations, the historical test values engine 122 may take rules and/or other considerations into account in determining if a test value substantially corresponds in time with, and should be included in a set with other test values. For example, certain blood tests like genetic markers, A1c for diabetes, and/or parathyroid hormone levels may be considered “valid” (e.g., based on standard medical practice) for several months and therefore those medical tests not repeated for several months. Based on this, the historical test values engine 122 may “carry forward” one or more of those test values and utilize those test values in one or more sets that include test values from more recent time periods. As one example, the historical test values engine 122 may identify, for a first set of test values and cost indications, one or more test values that resulted from one or more medical tests performed on 1/1/15, including a test value of A1c for diabetes that resulted from a medical test performed on 1/1/15. The historical test values engine 122 may further identify, for a second set of test values and cost indications, one or more test values that resulted from one or more medical tests performed on 2/1/15—and the test value of A1c for diabetes that resulted from the medical test performed on 1/1/15 (e.g., based on there not being a more recent test value of A1c for diabetes and 1/1/15 still being within a valid timeframe). Thus, the historical test values engine 122 may include certain test values in multiple sets of test values and cost indications for a patient identifier.

Generally, the regression correlation coefficients determination engine 123 calculates regression correlation coefficients for the medical test results based on the sets of historical test values and cost indicators compiled by the historical test values engine 122. The regression correlation coefficients are calculated using the test values as independent variable values and using the corresponding cost indications as dependent variable values. For example, the first test value that resulted from medical tests performed on 11/1/14 may be used as an independent variable and the cost indication that indicates less than a high cost threshold for medical care may be the dependent variable for that independent variable. Additional independent variables and corresponding dependent variables may be identified based on other values from the same patient identifier and/or other patient identifiers. As another example, where multiple test values are included in individual of the sets of historical test values and cost indicators compiled by the historical test values engine 122, the multiple test values from a set may be used as independent variable values and the cost indication of the set may be the dependent variable for those independent variables. Additional independent variables and corresponding dependent variables may be identified based on other values from set(s) from the same patient identifier and/or other patient identifiers.

The calculated regression correlation coefficients are associated with respective of the medical test results of the independent variables and each provide an indication of the relationship of the medical test result to a high cost for medical care. The regression correlation coefficients determination engine 123 may utilize various statistical regression techniques in calculating the regression correlation coefficients. For example, linear regression, logistic regression, and/or machine learning techniques may be utilized. In some implementations, the regression correlation coefficients determination engine 123 may comprise a statistical software package that receives the sets of historical test values and cost indicators compiled by the historical test values engine 122 and returns the regression correlation coefficients.

The historical analysis system 120 assigns the calculated regression correlation coefficients to respective medical test results and to the medical diagnosis. For example, the historical analysis system 120 may create a database entry in regression correlation coefficients database 156 that defines a triple that includes the regression correlation coefficient(s), the medical test result(s), and the medical diagnosis (and optionally one or more of the optional mandatory matching parameters). In some implementations, the historical analysis system 120 may index the regression correlation coefficients of regression correlation coefficients database 156 to enable more efficient retrieval of the regression correlation coefficients by the display generation system 140. For example, the regression coefficients database 156 may include a plurality of entries, with each entry being associated with a medical diagnosis and including regression correlation coefficients and indications of medical test results with which the regression correlation coefficients are associated. The historical analysis system 120 may generate an index of the entries based on one or more properties of the entries. For instance, the index may include one or more values associated with each entry, wherein the values each indicate the medical diagnosis associated with a respective entry. Accordingly, in determining regression correlation coefficients for a particular medical diagnosis, the display generation system 140 may more efficiently locate the regression correlation coefficients in regression correlation coefficients database 156 by utilizing the index to identify those entries indexed with the particular medical diagnosis. In some implementations, the historical analysis system 120 may additionally and/or alternatively assign the regression correlation coefficients in code executable by the overall HRS engine 141 and/or organ HRSs engine 142 in determining overall health risk scores and/or organ health risk scores as described herein.

Turning to FIG. 6, a flow chart is illustrated of an example method of calculating a regression correlation coefficient for a medical test result for a medical diagnosis. Other implementations may perform the steps in a different order, omit certain steps, and/or perform different and/or additional steps than those illustrated in FIG. 6. For convenience, aspects of FIG. 6 will be described with reference to a system of one or more computers, which may be located in disparate geographic facilities and are switched by the program engine to perform the process. The system may include, for example, one or more of the engines 121-123 of historical analysis system 120.

At step 600, a medical diagnosis and at least one medical test result are identified. For example, the system may identify a medical diagnosis of thyroid cancer and a medical test result of thyroid hormone levels (T3/T4). In some implementations, multiple medical test results may be identified (e.g., thyroid stimulating hormone, body mass index, and waist circumference) and/or the matching criteria may be defined more particularly, such as matching criteria that require a medical diagnosis of thyroid cancer and an extent of the thyroid cancer to be Stage II.

At step 605, a set of patient identifiers each associated with a value that indicates presence of the medical diagnosis and associated with a test value for the medical test result is identified from an electronic database such as patient data database 154 and/or another database with historical patient data. For example, where the medical diagnosis is thyroid cancer and the medical test result is thyroid hormone levels (T3/T4), the system may identify a random set of patient identifiers that are associated with a “true” value for the medical diagnosis and a test value for Thyroid hormone levels that optionally substantially corresponds in time (e.g., based on a timestamp or other data) with the value for the medical diagnosis (e.g., the time of the medical diagnosis and the time that the medical test are sufficiently close).

At step 610, independent variable values are determined for an analysis set based on the test values for the medical test result. For example, where the system identifies a medical test result of thyroid hormone levels serum electrolytes, actual test values associated with medical test results may be determined for the set of patient identifiers identified at step 605, and utilized as the independent variable values. In some implementations, the independent variable values may be normalized by the system and/or may already be normalized in a database from which the system retrieves the independent variable values.

At step 615, dependent variable values are determined for an analysis set based on, for example, medical costs incurred within a threshold time period of the test values for the medical test results. For example, for those patient identifiers identified at step 605, medical costs incurred within a threshold time period of the test values for medical test results determined at step 610 may be determined and compared to a high cost threshold, and an indication of whether the high cost threshold is satisfied paired with corresponding independent variable values. For example, where the system identifies a medical test result of thyroid hormone levels serum electrolytes, actual test values associated with medical test results may be determined at step 610 and each of the actual test values paired with a dependent variable that indicates whether medical costs that exceed a high cost threshold were incurred within one month (or other time frame) of the corresponding medical test result of thyroid hormone levels serum electrolytes.

At step 620, a regression correlation coefficient is calculated based on the analysis set. The regression correlation coefficient is associated with the independent variable and provides an indication of the relationship of test values for the medical test result to a high cost for medical care, such as a predetermined high cost for medical care based on previously described national standard database(s). The system may utilize various statistical regression techniques in calculating the regression correlation coefficient.

At step 625, the regression correlation coefficient is assigned to the medical test result and the medical diagnosis. For example, the system may create a database entry that defines a triple that includes the regression correlation coefficient, the medical test result, and the medical diagnosis.

In some implementations, the medical test results for which the historical analysis system 120 calculates regression correlation coefficients for a medical diagnosis include those that define a disease stage/progression, an illness severity, an illness complexity, and an illness volatility. In some implementations, the regression correlation coefficient(s) that define the disease stage/progression include the coefficient(s) for medical test result(s) that are utilized to primarily determine an extent of the medical diagnosis such as one or more specific stages of a medical diagnosis. As one example, for kidney disease the estimated glomerular filtration rate (e-GFR) would be the medical test result that defines the disease stage/progression. Other medical test results would determine the extent of other medical diagnoses.

In some implementations, the regression correlation coefficient(s) that define the illness severity include a coefficient that is determined based on a ratio between the test values for medical test result(s) of serum Blood Urea Nitrogen (BUN) and serum creatinine. Such ratio test values further define severity of an illness to a greater extent than the single test results used to primarily diagnose the primary illness. In some instances, the ratio between serum BUN and serum creatinine may be a medical test result for which test values are provided in patient data database 154. In other instances, the historical analysis system 120 may calculate the ratio based on BUN and serum creatinine values that are provided in patient data database 154. For example, the historical values engine 122 may calculate those test values for identified patient identifiers and use those test values as independent variables in determining a regression correlation coefficient for a medical test result that defines the ratio between serum BUN and serum creatinine.

In some implementations, the regression correlation coefficient(s) that define the illness volatility include a coefficient that is determined based on fluctuation over time between the z-scores of medical test result(s) for serum creatinine and serum BUN. The fluctuation over time may be, for example, the fluctuation over the last X medical tests or the fluctuation since the patient was diagnosed with the medical diagnosis. In some instances, the fluctuation over time between serum BUN and serum Creatinine may be a medical test result for which test values are provided in patient data database 154. In other instances, the historical analysis system 120 may calculate the fluctuation based on serum BUN and serum creatinine values that are provided in patient data database 154. For example, the historical values engine 122 may calculate those test values for identified patient identifiers and use those test values as independent variables in determining a regression correlation coefficient for a medical test result that defines the ratio between serum BUN and serum creatinine.

In some implementations, the regression correlation coefficient(s) that define the illness complexity include coefficients for all routine medical laboratory test results with the exception of those test results that define the medical diagnosis. For example, where the medical diagnosis is thyroid cancer the regression correlation coefficient(s) may be based on medical test results that indicate one or more of: complete blood counts with white cell differential, serum electrolytes, liver profile enzymes, metabolic study values, estimated glomerular filtration rate, selected tumor markers, NCI (National Cancer Institute) identified genetic tumor markers, lipid and tri-glyceride values, total cholesterol and ratio high-density lipoprotein (HDL) & low-density lipoprotein (LDL), blood pressure, body mass index, waist circumference, and/or patient health questionnaire-9 (PHQ-9) results.

In some implementations, the historical analysis system 120 further assigns one or more (e.g., all) of the regression correlation coefficients to an indication of the organ that is measured by the medical test result. For example, medical test results for serum creatinine, blood urea nitrogen, potassium, and micro-albumin in urine measure function of the kidney. Accordingly, the historical analysis system 120 may assign the regression correlation coefficients for those medical test results to an indication of the kidney. As another example, medical test results for bilirubin, alkaline phosphatase, aspartate transaminase (AST), and alanine transaminase (ALT) measure function of the liver. Accordingly, the historical analysis system 120 may assign the regression correlation coefficients for those medical test results to an indication of the liver. Other regression correlation coefficients may be assigned to respective organs based on medical literature and/or other resource that identifies specific lab tests employed to measure that organ's function. As described herein, the display generation system 140 may utilize one or more regression correlation coefficients associated with medical test results that indicate the health of a particular organ and the test values for that organ for a patient, to calculate an organ health risk score for that organ.

Generally, the display generation system 140 calculates an overall health risk score (also referred to herein as “overall HRS”) and individual organ health risk scores (also referred to herein as “organ HRSs”) based on patient data of a patient identifier and generates an electronic display that provides indications of the calculated overall health risk score and individual organ health risk scores. In some implementations, the organ health risk scores are utilized to determine display properties of graphical representations of corresponding organs and the graphical representations are provided in the electronic display with the determined display properties. In some of those implementations, the electronic display includes an indication of the overall health risk score and an indication of an avatar for the patient with the graphical representations (with the display properties) provided in anatomically appropriate positions in the avatar.

The display generation system 140 may further receive input from one of the user interface input device(s) 104 and modify the electronic display based on the received input. For example, the display generation system 140 may initially provide an electronic display with display properties of graphical representations of corresponding organs determined based on most recent in time test values for a patient identifier and input from one of the user interface input device(s) 104 may indicate a request for the display to be updated based on less recent in time test values. Based on receiving the input, the display generation system 140 may alter the display by altering the indication of the overall health risk score based on a calculated overall health risk score based on the less recent in time test values and/or altering one or more of the display properties of the organs based on calculated organ health risk scores based on the less recent in time test values. In some implementations, the display generation system 140 may optionally receive therapy input from one of the user interface input device(s) 104 and/or biometric data input device(s) 108 and calculate predicted overall HRS and/or predicted organ HRSs based on the therapy input. The display generation system 140 may alter the display by altering the indication of the overall health risk to reflect the predicted overall HRS and/or altering one or more of the display properties of the organs to reflect predicted organ HRSs.

In various implementations display generation system 140 may include an overall HRS engine 141, an organ HRSs engine 142, a display generation engine 143, and/or a therapy input engine 144. In some implementations, all or aspects of engines 141, 142, 143, and/or 144 may be omitted. In some implementations, all or aspects of engines 141, 142, 143, and/or 144 may be combined. In some implementations, all or aspects of engines 141, 142, 143, and/or 144 may be implemented in a component that is separate from display generation system 140

Generally, overall HRS engine 141 calculates overall health risk scores for patient identifiers. The overall HRS engine 141 calculates an overall health risk score for a patient identifier for a given time period based on applying test values for medical test results of the given time period (e.g., test values for medical tests conducted on the same day, or within a threshold time period of one another) to regression correlation coefficients for those medical test results for a medical diagnosis for the patient identifier. In some implementations, application of the test values to the regression correlation coefficients results in a value that indicates probability of increased need for high-cost medical care. In some of those implementations, the probability is utilized as the overall health risk score or as the basis for the overall health risk score (e.g., the probability is converted to a value on a 1 to 5 or 1 to 10 scale).

As one example, patient data for a patient identifier may be received from patient data database 154 and/or another source. The overall HRS engine 141 may identify a medical diagnosis of Medical Condition 1 based on the patient data for the patient identifier. The overall HRS engine 141 may identify regression correlation coefficients for medical test results for the medical diagnosis from regression coefficients database 156. For example, a vector of {(0.01, MTR1); (0.08, MTR2); (0.1, MTR3); (0.01, MTR4); (0.15, MTR5); (0.12, MTR6); (0.19, MTR7); (0.07, MTR8); . . . (0.22, MTRn)} may be identified, wherein the numerical values indicate the regression correlation coefficients and the “MTR” values indicate a medical test result. As described herein, the regression correlation coefficients are statistically calculated based on historical data and each generally indicate the calculated impact, for a particular medical diagnosis (and optionally other factor(s)), of values associated with a respective medical test result.

The overall HRS engine 141 may further identify, based on the patient data for the patient identifier, most recent test values for the medical test results indicated in the vector of values identified from regression coefficients database 156. The test values for the patient data may be represented as the vector {(0, MTR1); (0, MTR2); (1, MTR3); (7, MTR4); (0, MTR5); (0, MTR6); (0, MTR7); (1, MTR8); . . . (1, MTRn)}. In the preceding vector, the values are normalized values (or for those values that indicate selected tumor markers and/or genetic markers “0” indicates lack of presence and “1” indicates presence). The values may be normalized by patient data processing system 130 and/or display generation system 140 as described herein. It is noted that in some implementations, one or more of the values may be calculated by the display generation system 140 based on the patient data for the patient identifier. For example, in some implementations the patient data for the patient identifier may include a test value for a medical test result of serum BUN and a test value for a medical test result for serum creatinine, and the display generation system 140 may calculate a normalized value for a ratio between the test values for serum BUN and serum creatinine.

The overall HRS engine 141 may calculate the overall health risk score based on the numerical values of the two vectors. For example, the overall HRS engine 141 may calculate the overall health risk score based on the dot product of the two vectors, optionally taking into account a constant (e.g., an intercept value) defined in the regression coefficients database 156 for the medical diagnosis. For example, the regression correlation coefficients may be for a logistic regression and the overall HRS engine 141 may calculate the overall health risk score based on applying the calculated dot product to the variable “lp” in the following equation: e^{lp}/(1+e^{lp}) to determine a probability, and using the probability as the basis for the overall health risk score. For example, the probability may be multiplied by ten, optionally rounded, and the resulting value utilized as the overall health risk score.

The overall HRS engine 141 may further identify, based on the patient data for the patient identifier, less recent in time test values for the medical test results indicated in the vector of values identified from regression coefficients database 156—and calculate overall health risk scores for those less recent in time test values. For example, the overall HRS engine 141 may calculate a second overall health risk score based on test values for medical test results from two months prior, a third overall health risk score based on test values for medical test results from three months prior, etc.

In some implementations, the regression coefficients and test values on which the overall HRS engine 141 calculates the overall health risk score comprise an illness severity component, an illness volatility component, an illness complexity component, and/or a disease stage/progression component. As described above, the regression correlation coefficient(s) that define the disease stage/progression include the coefficient(s) for medical test result(s) that are utilized to primarily determine an extent of the medical diagnosis. The regression correlation coefficient(s) that define the illness severity include a coefficient that is determined based on a ratio between certain combinations of test values for example, medical test result(s) of serum Blood Urea Nitrogen (BUN) and serum creatinine. The regression correlation coefficient(s) that define the illness volatility include a coefficient that is determined based on fluctuation over time of the z-scores of certain medical test result(s) which are determined to be significant for organ health, for example serum creatinine and serum BUN for kidney function. The regression correlation coefficient(s) that define the illness complexity include coefficients for all routine medical laboratory test results with the exception of those test results that define the key diagnostic test for a medical diagnosis, for example e-GFR and serum creatinine for diagnosis of chronic kidney disease (CKD).

In some implementations, the overall HRS engine 141 calculates scores for the illness severity component, the illness volatility component, the illness complexity component, and/or the disease stage/progression component. The overall HRS engine 141 may calculate the score for a given component for a patient identifier based on applying the regression coefficients for the given component to the test values of the patient identifier for the medical test results associated with the regression coefficients for the given component. For example, to determine a score for the illness volatility component, the coefficient for illness volatility may be multiplied by a normalized test value that is determined based on fluctuation over time between the z-scores of medical test result(s) for serum creatinine and serum BUN for the patient identifier. Where the regression correlation coefficients are for a logistic regression the overall HRS engine 141 may calculate the score for the illness volatility component based on applying the product of the preceding multiplication to the variable “lp” (lp=logistic predictor) in the following equation: e^{lp}/(1+e^{lp}) to determine a probability, and use the probability as the basis for the score for the illness volatility component. For example, the probability may be multiplied by 5, optionally rounded, and the resulting value utilized as the score for the illness volatility component.

Generally, organ HRSs engine 142 calculates individual organ health risk scores for patient identifiers. The organ HRSs engine 142 calculates an individual organ health risk score for an organ of a patient identifier for a given time period based on applying one or more selected test values for one or more medical test results (for the organ) of the given time period (e.g., test values for medical tests conducted on the same day, or within a threshold time period of one another) to regression correlation coefficients for those one or more medical test results (for the organ) for a medical diagnosis for the patient identifier. In some implementations, application of the test value(s) to the regression correlation coefficient(s) results in a value that indicates the probability for increased need of medical care based on the organ. In some of those implementations, the probability is utilized as the individual organ health risk score or as the basis for the individual organ health risk score (e.g., the probability is converted to a value on a 1 to 5 or 1 to 10 scale).

As one example, and continuing with the example above, the organ HRSs engine 142 may identify regression correlation coefficients for medical test results for the medical diagnosis from regression coefficients database 156. For example, the vector of {(0.01, MTR1); (0.08, MTR2); (0.1, MTR3); (0.01, MTR4); (0.15, MTR5); (0.12, MTR6); (0.19, MTR7); (0.07, MTR8); . . . (0.22, MTRn)} may be identified, wherein the numerical values indicate the regression correlation coefficients and the “MTR” values indicate a medical test result. The organ HRSs engine 142 may further identify, based on the patient data for the patient identifier, most recent test values for the medical test results indicated in the vector of values identified from regression coefficients database 156. The test values for the patient data may be represented as the vector {(0, MTR1); (0, MTR2); (1, MTR3); (7, MTR4); (0, MTR5); (0, MTR6); (0, MTR7); (1, MTR8); . . . (1, MTRn)}.

Data of regression coefficients database 156 and/or logic of organ HRSs engine 142 may indicate that MTR1 is a medical test result associated with function of the pancreas, MTR2 and MTR3 are medical test results associated with function of the liver, etc. To calculate the pancreas health risk score, the HRSs engine 142 may apply the regression coefficient (0.01) for the pancreas medical test result (MTR1) to the test value for that medical test result (0). Similarly, to calculate the liver health risk score, the organ HRSs engine 142 may apply the regression coefficients (0.08, 0.1) for the liver medical test results (MTR2, MTR3) to the test value for that medical test result (0, 1). Where the regression correlation coefficients are for a logistic regression the organ HRSs engine 142 may calculate the score for a given organ based on taking the dot product of the regression correlation coefficients for the medical test results and the test values for the medical test results, and applying the dot product to the variable “lp” in the following equation: e^{lp}/(1+e^{lp}) to determine a probability, and use the probability as the basis for the score for the given organ. For example, the probability may be multiplied by 10, optionally rounded, and the resulting value utilized as the score for the given organ.

The organ HRSs engine 142 may further identify, based on the patient data for the patient identifier, less recent in time test values for the medical test results indicated in the vector of values identified from regression coefficients database 156—and calculate individual organ health risk scores for those less recent in time test values. For example, the organ HRSs engine 142 may calculate a second set of organ health risk scores based on test values for medical test results from two months prior, a third set of organ health risk scores based on test values for medical test results from three months prior, etc.

Generally, the display generation engine 143 generates an electronic display of a human avatar with display properties that are particularized to one or more of the scores calculated by the overall HRS engine 141 and/or organ HRSs engine 142. The electronic display may be provided to a user via one or more user interface output devices 102, such as a display screen of a tablet computing device, a monitor of a desktop or laptop computing device, a display screen on glasses or other wearable computing device, a holographic projector, etc. The display properties may include display properties for graphical representations of human organs to be presented in the electronic display in combination with the human avatar. Each of the display properties may be based on a magnitude of an organ health risk score that is calculated for a corresponding organ by the organ HRSs engine 142. For example, a display property of a graphical representation of a pancreas may be a color of “red” based on an organ health risk score for the pancreas indicating a large degree of dysfunction of the organ; whereas a display property of a graphical representation of a heart may be a color of “yellow” based on a health risk score for the heart indicating a mild degree of dysfunction of the organ. In some implementations, the display properties may also include an indication of an overall health risk score calculated by the overall HRS engine 141.

In some implementations, an overall health risk score and/or individual organ health risk scores may be calculated for each of a plurality of time periods and the display generation engine 143 may modify the electronic display to illustrate changes to the health risk scores and/or individual organ health risk score over the time periods. For example, the display generation engine 143 may receive time period adjustment inputs from the user interface input device 104 to switch among multiple time periods and the display generation engine 143 may update the electronic display based on the health risk score and/or individual organ health risk scores for the appropriate time periods. For instance, a health risk score and organ health risk scores may be provided in the electronic display for a “current” time period then, in response to a time period adjustment input, a health risk score and organ health risk scores for a “past” or “future” time period may be provided in the electronic display.

Generally, the therapy input engine 144 receives therapy input from one or more of the user interface input device(s) 104, one or more of the biometric input device(s) 108, and/or other sources. The therapy input is indicative of actions performed and/or performable by a patient associated with a patient identifier and the therapy input engine 144 utilizes the therapy input to determine predicted changes to test values of the patient identifier that may result based on the therapy input. The therapy input engine 144 provides the predicted changes to the overall HRS engine 141 and/or the organ HRSs engine 142 to determine one or more adjusted overall HRSs and/or one or more adjusted organ HRSs based on those predicted changes.

For example, one of the biometric data input devices 108 may be a wearable fitness monitor that measures steps of a patient or otherwise measures activity of a patient and the therapy input engine 144 may automatically receive data from the biometric data input device 108 that indicates a level of activity over a time period (e.g., for a day, for each of multiple days, for a week). The therapy input engine 144 may utilize the data to determine impacts on test values for a patient identifier of the patient based on therapy adjustment values database 158. For example, therapy adjustment values database 158 may indicate that moderate physical exercise for a period of 8 weeks may reduce A1c levels by 0.6 percent, may reduce serum triglycerides by 22.1 mg/dl, and/or reduce BMI by a certain percentage. Predicted adjustments for one or more test values that are included in the therapy adjustment values database 158 may be based on, for example, one or more medical studies and/or historical statistical analysis of previously monitored activities and resulting adjustments to test values. Based on data from the biometric data input device 108 that indicates the patient identifier engaged in moderate physical exercise for a period of 8 weeks, the therapy input engine 144 may reduce most recently measured A1c levels of the patient by 0.6 percent, may reduce most recently measured serum triglycerides by 22.1 mg/dl, and/or reduce a most recently measured BMI by a certain percentage and send the reduced values to the overall HRS engine 141 and/or the organ HRSs engine 142 to determine one or more adjusted overall HRSs and/or one or more adjusted organ HRSs based on those predicted changes. The display generation engine 143 may further adjust (or enable adjustment of) the electronic display to provide indications of one or more adjusted overall HRSs and/or one or more adjusted organ HRSs.

As another example, based on data from the biometric data input device 108 that indicates the patient identifier engaged in moderate physical exercise for a period of 1 week, the therapy input engine 144 may reduce most recently measured A1c levels of the patient by a lesser percentage (e.g., 0.08 percent), may reduce most recently measured serum triglycerides by a lesser amount (e.g., 2.75 mg/dl), and/or reduce a most recently measured BMI by a lesser percentage and send the adjusted values to the overall HRS engine 141 and/or the organ HRSs engine 142 to determine one or more adjusted overall HRSs and/or one or more adjusted organ HRSs based on those predicted changes. Moreover, the therapy input engine 144 may determine predicted future adjusted test values based on assuming the same level of exercise continues for 8 weeks, or other time period, and send the adjusted values to the overall HRS engine 141 and/or the organ HRSs engine 142 to determine one or more predicted future overall HRSs and/or one or more predicted future organ HRSs based on those predicted changes.

As yet another example, a user may utilize one of the user interface input devices 104 to provide therapy input that indicates actions performed or performable by the patient such as biometric data that indicates actual or anticipated: heart rate, dietary calorie values, body mass index (“BMI”), activity values, and/or sleep values. The therapy input engine 144 may adjust one or more test values based on the input, send the adjusted values to the overall HRS engine 141 and/or the organ HRSs engine 142 to determine one or more adjusted overall HRSs and/or one or more adjusted organ HRSs based on those predicted changes. For instance, the user may provide therapy input that indicates the user intends to consume no more than 2500 calories per day for the next two months and exercise at least 30 minutes per day for the next two months. The therapy input engine 144 may determine adjusted test values based on assuming those calorie and exercise inputs for the next two months, and send the adjusted values to the overall HRS engine 141 and/or the organ HRSs engine 142 to determine a predicted future overall HRS and/or one or more predicted adjusted future HRSs for two months in the future.

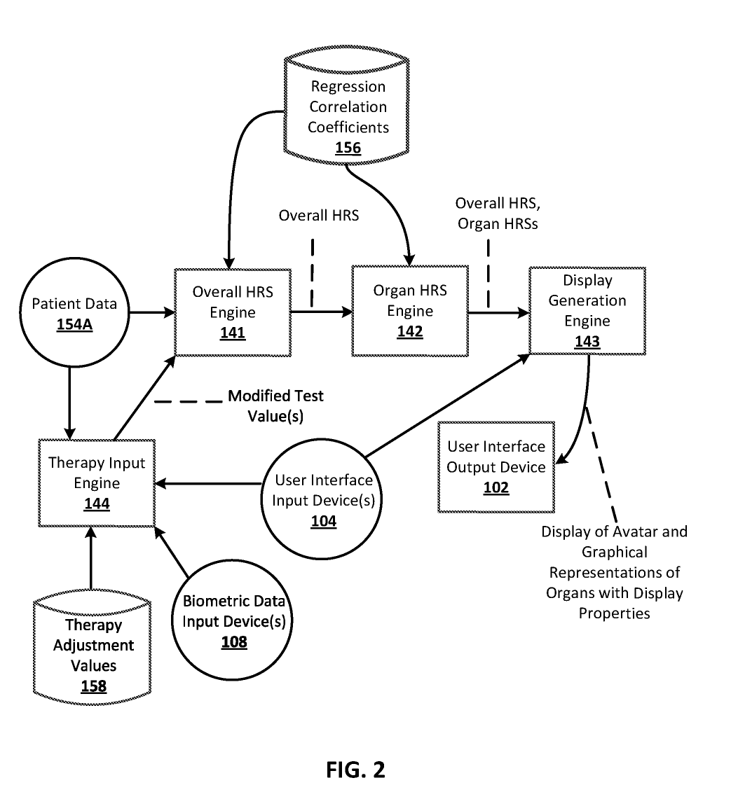

Additional description of the display generation system 140 is provided below with reference to FIGS. 2-6 and 7-9.

In various implementations, a user may interact with the display generation system 140 via a computing device that includes one of the user interface input devices 104, one of the user interface output devices 102, and optionally includes one or more (e.g., all) aspects of the display generation system 140 itself. While the user may operate a plurality of computing devices, for the sake of brevity, examples described in this disclosure will focus on the user operating a single computing device. Moreover, while in some implementations multiple users may interact with the display generation system 140 via multiple client devices (e.g., when all or aspects of the display generation system 140 operate on a remote server accessible to a plurality of computing devices via the network(s) 101), for the sake of brevity, examples described in this disclosure will focus on a single user operating a computing device.

In various implementations, the computing device via which a user interacts with the display generation system 140 includes one or more applications to facilitate the sending and receiving of data over a network, to enable presentation (e.g., display) of data received from the display generation system 140, and/or enable data to be sent to the display generation system 140 (e.g., feedback related to the electronic display, therapy input). For example, the computing device may execute one or more applications, such as a browser or stand-alone application, that may render one or more of the electronic displays described herein and/or that may receive input from one or more user interface input devices of the computing device and provide data to the display generation system 140 based on such input.

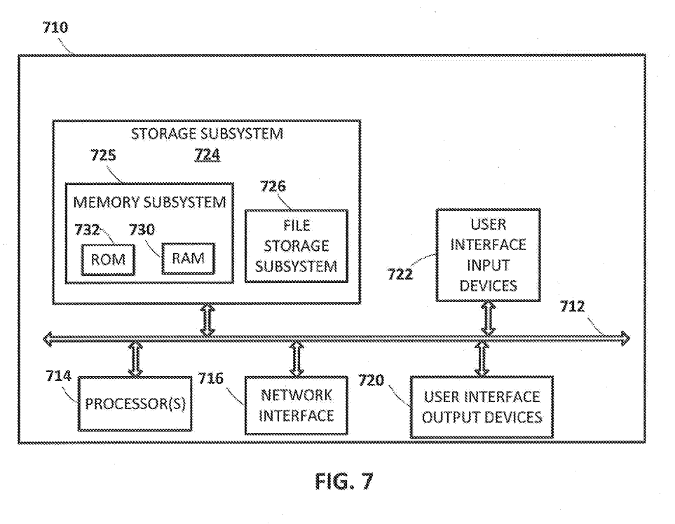

The components of the example environment of FIG. 1 may each include memory for storage of data and software applications, a processor for accessing data and executing applications, and components that facilitate communication over a network. In some implementations, such components may include hardware that shares one or more characteristics with the example computer system that is illustrated in FIG. 7. The operations performed by one or more components of the example environment may optionally be distributed across multiple computer systems. For example, the steps performed by the display generation system 140 may be performed via one or more computer programs running on one or more servers in one or more locations that are coupled to each other through a network.

FIG. 2 illustrates an example of calculating an overall health risk score and an organ health risk scores based on patient data, generating an electronic display with display properties determined based on the calculated scores, and adjusting the electronic display based on user input and/or therapy input.

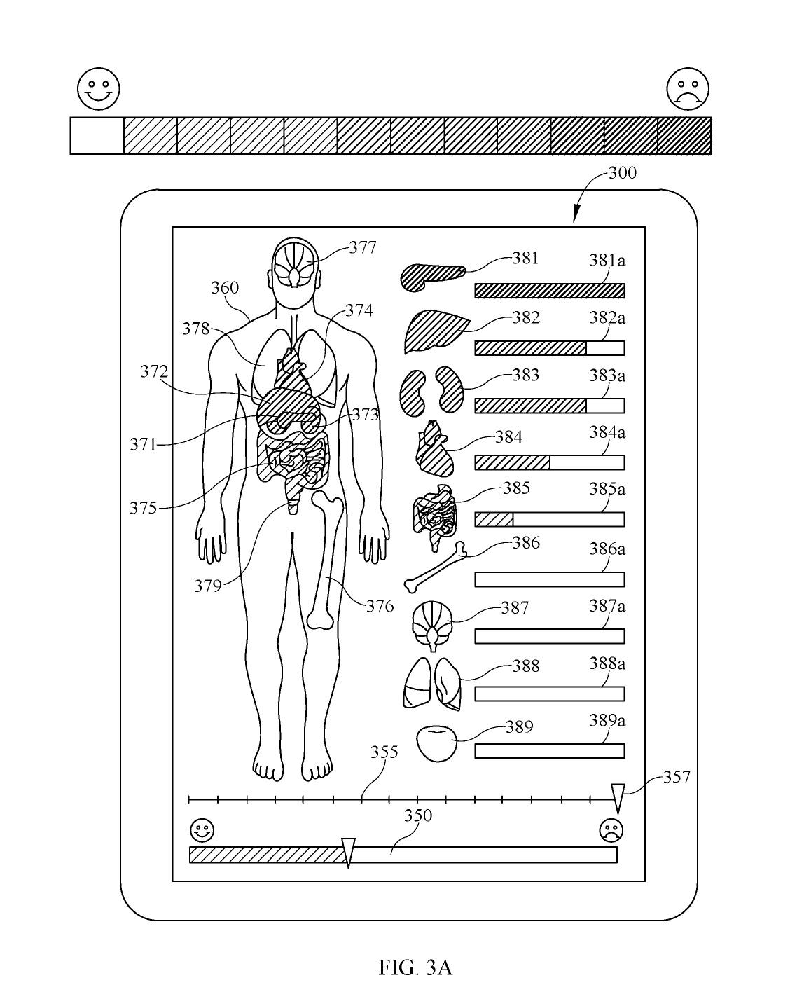

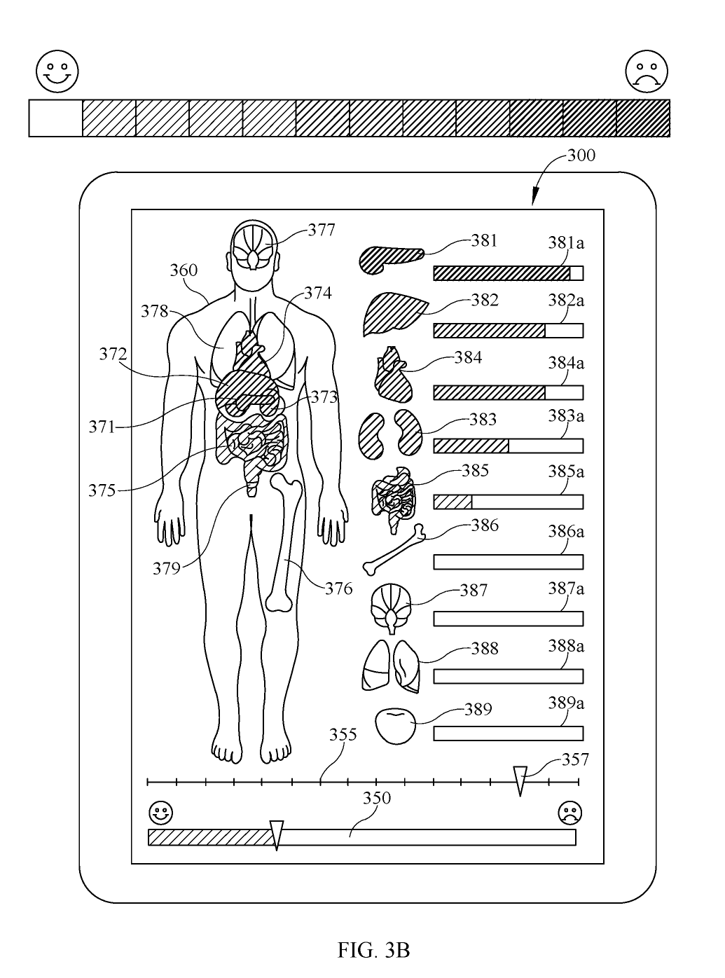

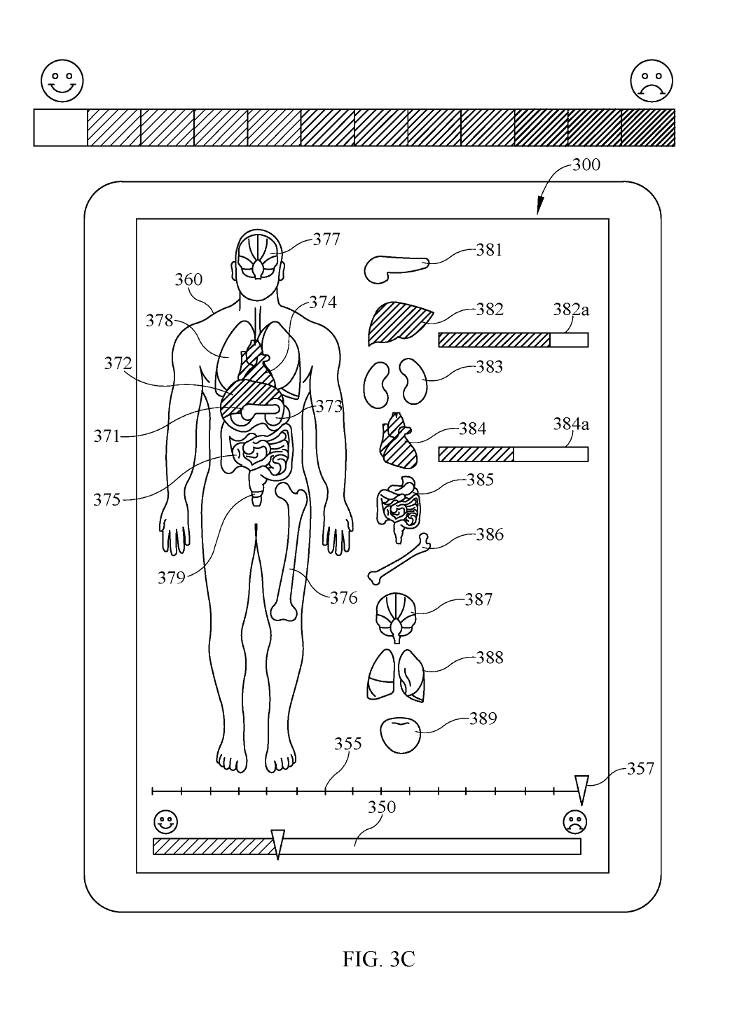

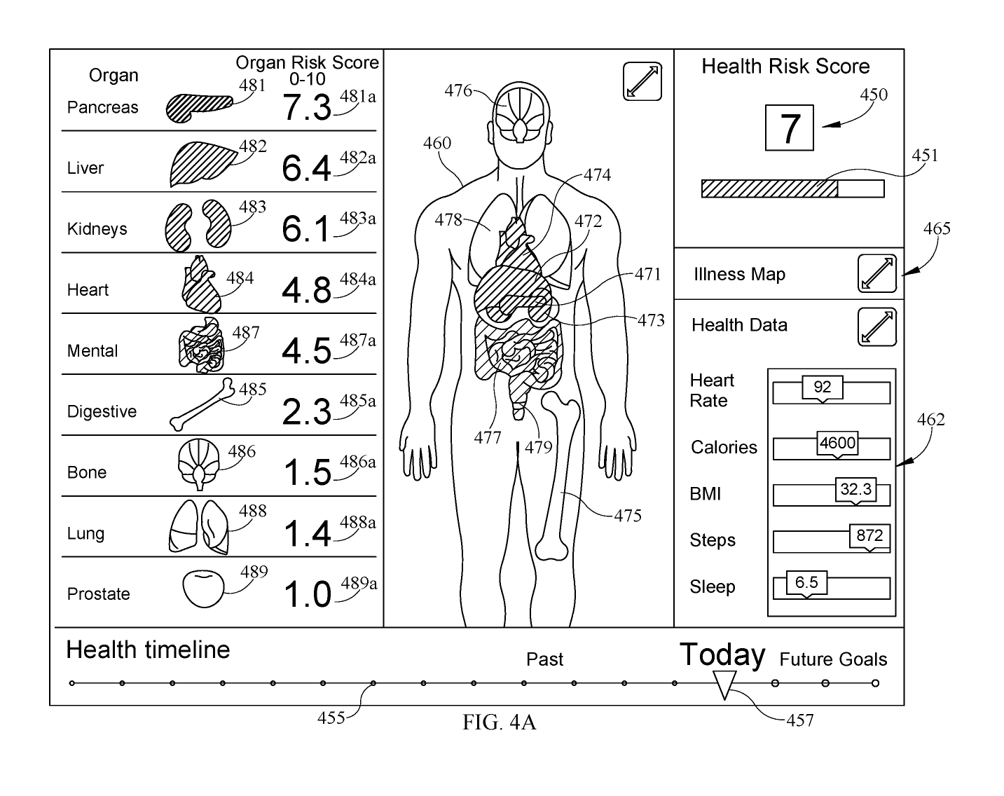

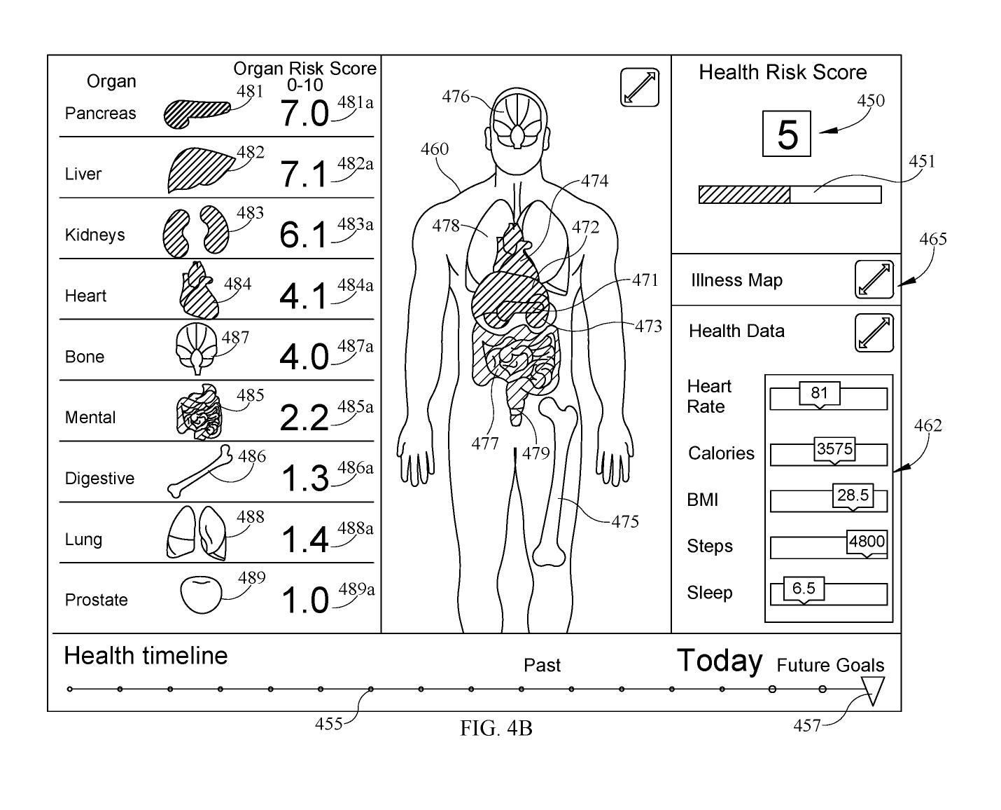

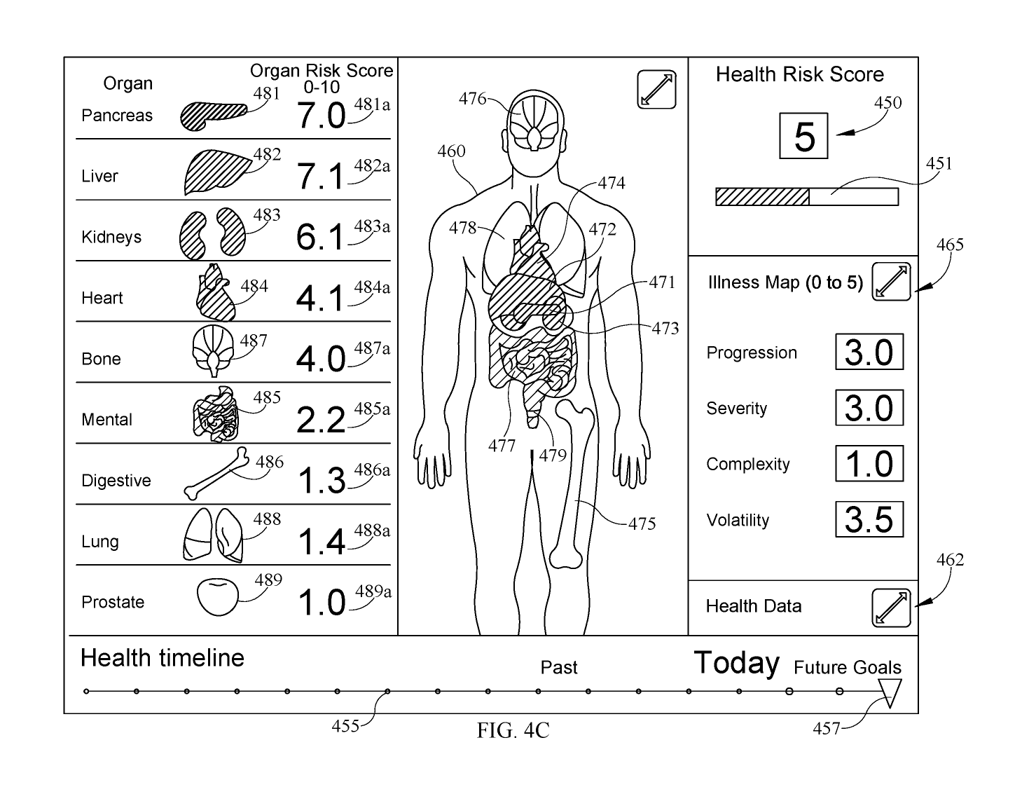

To aid in explaining the example of FIG. 2, it will be described in the context of a patient identifier having CKD and will be explained with reference to FIGS. 3A-4C.

Overall HRS engine 141 receives patient data 154A for a patient identifier of a patient. The patient data defines a medical diagnosis for the patient identifier and test values for medical test results of the patient identifier. For example, the patient data may identify an ICD value that indicates the patient has CKD. The overall HRS engine 141 identifies regression coefficients for CKD from the regression coefficients database 156. In some implementations, the overall HRS engine 141 may identify regression coefficients for CKD and for one or more additional mandatory criteria such as criteria that define one or more extents of the CKD medical diagnosis (e.g., Stage II or Stage III).

The overall HRS engine 141 uses the regression coefficients for the medical diagnosis and the test values for the patient identifier to calculate an overall health risk score for the patient identifier for each of one or more time periods. For example, the overall HRS engine 141 may apply test values for medical test results of a first time period to the regression correlation coefficients to determine a first overall health risk score for the first time period, may apply test values for medical test results of a second time period to the regression correlation coefficients to determine a second overall health risk score for the second time period, etc.

Organ HRSs engine 142 calculates organ health risk scores for organs of the patient identifier for each of one or more time periods. The organ HRSs engine 142 calculates an individual organ health risk score for an organ of the patient identifier for a given time period based on applying one or more selected test values for one or more medical test results (for the organ) of the given time period to the regression correlation coefficients for those one or more medical test results (for the organ). For example, the organ HRSs engine 142 may determine, for a first time period, a first organ risk score for the heart, a first organ risk score for the liver, a first organ risk score for the pancreas, etc.; determine, for a second time period, a second organ risk score for the heart, a second organ risk score for the liver, a second organ risk score for the pancreas, etc.; and so forth. The organ HRSs engine 142 may receive the test values directly or from the overall HRS engine 141. Also, the organ HRSs engine 142 may receive the regression correlation coefficients from the regression correlation coefficients database 156 or the overall HRS engine 141.

The overall health risk score for each of the one or more time periods and the organ health risk scores for each of the one or more time periods are provided to the display generation engine 143. The display generation engine 143 generates an electronic display of a human avatar with an indication of the overall health risk score for one of the time periods and graphical representations of one or more of the organs with display properties that are particularized to one or more of the organ health risk scores for the time period. The electronic display may be provided to a user via the user interface output device 102.

FIG. 3A illustrates an example of an electronic display 300 that may be generated by the display generation engine 143. The electronic display 300 includes an avatar 360 for a patient with graphical representations of organs 371-379 provided in anatomically appropriate positions in the avatar 360. The graphical representations of organs 371-379 include graphical representations of: a pancreas 371, a liver 372, kidneys 373, a heart 374, a gastrointestinal tract 375, a bone 376, a brain 377, a lung 378, and a prostate 379. In other implementations, more or fewer graphical representations of organs may be displayed, including additional organs such as ovaries not displayed in the example of FIG. 3A.

The display generation engine 143 has determined display properties for each of the graphical representations of the organs 371-379 based on a magnitude of respective organ health risk scores for the organs at a given time period. In particular, in FIG. 3A the shading applied to each of the graphical representations of the organs 371-379 is determined based on the magnitude of the respective organ health risk scores, with no shading being indicative of an organ health risk score with no degree of dysfunction/most unlikely to lead to the need for increased levels of medical care and/or costs, with increasingly darker shading indicating health risk scores with increasing degree of dysfunction/increasingly likely to lead to increased medical care and costs, with the greatest amount of shading being indicative of an organ health risk score with the highest degree of dysfunction/most likely to lead to increased need for medical care and costs. This is indicated in the legend 1 depicted at the top of FIG. 3A that illustrates the various shadings moving from no shading (left most of the legend 1) to the greatest amount of shading (right most of the legend 1). The shading selected by the display generation engine 143 for a particular graphical representation of one of the organs 371-379 may be based on, for example, an electronic mapping between the organ health risk score for the organ and the shading.

Although different shadings are illustrated in FIG. 3A, alternative display properties may be determined by the display generation engine 143 to illustrate the different organ health risk scores. For example, different colors may be used instead of shading such as a color scale that moves from green (no dysfunction/least likely to predict increased need for medical care and costs), to yellow (some dysfunction/somewhat likely to predict increased need for medical care and costs), to orange (more dysfunction/more likely to lead to increased medical care and costs), to red (most dysfunction/most likely to lead to predict high requirement for increased medical care, hospitalization and increased costs), optionally with gradients of colors there between. For example, where color is used instead of (or in addition to) shading, an organ health risk score that indicates a 0% to 30% probability of requiring increased medical care may be mapped to “green”, an organ health risk score that indicates a 31% to 50% probability of requiring increased medical care may be mapped to “yellow”, an organ health risk score that indicates a 51% to 70% probability of requiring increased medical care may be mapped to “orange”, and an organ health risk score that indicates a 71% to 100% probability of requiring increased medical care may be mapped to “red”. Also, although graphical representations of organs that have organ health risk scores indicative of no dysfunction (organs 376-379) are illustrated in the avatar, in other implementations organs with organ health risk scores indicative of no dysfunction (or less than a threshold level of dysfunction) may be omitted from being graphically represented in the avatar 360.

The electronic display 300 also includes additional graphical representations of organs (381-389) provided to the right of the avatar 360. The additional graphical representations of organs 381-389 include graphical representations of: a pancreas 381, a liver 382, kidneys 383, a heart 384, a gastrointestinal tract 385, a bone 386, a brain 387, a lung 388, and a prostate 389. In other implementations, more or fewer (e.g., none) additional graphical representations of organs may be displayed, including additional organs not displayed in the example of FIG. 3A. The display generation engine 143 has determined display properties for each of the additional graphical representations of the organs 381-389 based on a magnitude of respective individual organ health risk scores for the organs at a given time period—and that match the display properties of the graphical representations of organs 371-379.

The electronic display 300 also includes additional display properties 381a-389a provided to the right of respective of the additional graphical representations of organs 381-389. Each of the additional display properties is a bar graph that provides a scalar indication of the individual organ health risk score of a corresponding of the organs—and is provided with shading that matches the shading of a corresponding of the additional graphical representations of the organs 381-389. This scalar representation depicts the relative impact for each organ's contribution to the total risk score. For example, the additional display property 381a provides a scalar indication of the individual organ health risk score for the pancreas and has a shading that matches the shading of the additional graphical representation of the pancreas 381.

The display generation engine 143 has ordered the additional graphical representations of organs 381-389 and the additional display properties 381a-389a based on the respective individual organ health risk scores. In particular, in FIG. 3A the additional graphical representation of the pancreas 381 is presented at the top based on the pancreas having the highest degree of dysfunction as indicated by is organ health risk score, the additional graphical representation of the liver 382 is presented next based on the liver having the next highest degree of dysfunction as indicated by is organ health risk score, etc.

The electronic display 300 also includes an overall health risk score bar graph 350 that provides a visual indication of the overall health risk score for the given time period. The overall health risk score bar graph 350 includes display properties determined by the display generation engine 143 that includes a scalar indication of the magnitude of the overall health risk score and shading that also indicates the magnitude. The shading of the overall health risk score bar graph 350 is based on the same scale as the shading of the graphical representations of the organs 371-379 (i.e., no shading being indicative of an overall health risk score most unlikely to lead to increased need for medical care and cost, with increasingly darker shading indicating increasingly likely to lead to increased need for medical care and costs, and with the greatest amount of shading being indicative of an overall health risk score most likely to lead to increased levels of care and costs).

The electronic display 300 also includes a time period adjustable user interface element 357 that indicates the display properties of the electronic display 300 are being displayed for the most recent time period (based on its rightmost position). As described in more detail below, the time period adjustable user interface element 357 may be adjusted along the timeline 355 to select a desired earlier time period and cause the display generation engine 143 to update the electronic display based on an overall HRS and organ HRSs from the earlier time period.

Referring again to FIG. 2, a user may utilize one or more user interface input device(s) 104 to provide input to the display generation engine 143, and the display generation engine 143 may alter one or more aspects of the display based on the input. As one example, the user may utilize one of the user interface input device(s) 104 to provide a time period adjustment input and the display generation engine 143 may alter the electronic display based on the time period adjustment input.

One example is provided with reference to FIG. 3B. FIG. 3B illustrates the example electronic display 300 of FIG. 3A, where the user has adjusted the time period adjustable user interface element 357 to an earlier time period. The display generation engine 143 receives an indication of the adjustment to the earlier time period and, in response to the adjustment, updates the electronic display 300 based on an overall HRS and organ HRSs from the earlier time period. In particular, the electronic display 300 has been modified to provide an indication in the overall health risk score bar graph 350 of an overall health risk score for the patient at the earlier time period that indicates a lesser likelihood of increased costs than the overall health risk score indicated by the overall health risk score bar graph 350 of FIG. 3A. Also, the display properties of the graphical representations of the pancreas 371 and kidneys 373 have been modified to reflect that the individual organ heal risk scores for the pancreas and kidneys indicate lesser likelihood of increased need for medical care and costs/less dysfunction for those organs. The display properties of the additional graphical representations of the pancreas 381 and kidneys 383 and corresponding bar graphs 381a and 381b have likewise been updated. Moreover, the display generation engine 143 has adjusted the order of the additional graphical representations of organs 381-389 to “swap” the positions of additional graphical representations 383 and 384 (relative to their positions in FIG. 3A) based on the individual organ health risk score for the kidneys being less indicative of dysfunction than that of the gastrointestinal tract at the previous time period of FIG. 3B.

With reference to FIG. 3C, another example is provided of a user utilizing one or more user interface input device(s) 104 to provide input to the display generation engine 143, and the display generation engine 143 altering one or more aspects of the display based on the input. FIG. 3C illustrates the example electronic display 300 of FIG. 3A that has been modified in response to a user selection of graphical representations of the liver and the heart. The user selection may be, for example, a selection of the graphical representations 372 and 374 and/or a selection of the additional graphical representations 382 and 384. For example, where the user interface input device 104 is a touch screen of a tablet computing device, the user may “tap” (e.g., a short tap, double tap, and/or long-tap) the graphical representation of the liver 382 and the graphical representation of the heart 384. Also, for example, the user may “swipe away” the other additional graphical representations of the organs 382, 383, and 385-389 to exclude them and thereby select the graphical representation of the liver 382 and the graphical representation of the heart 384. Other selection techniques and user interface input devices 104 may be utilized.

Regardless of the selection technique, in response to the user selection of graphical representations of the liver and the heart, the display generation engine 143 modifies the additional graphical representations of the non-selected organs 382, 383, and 385-389 and the graphical representations of the non-selected organs 371, 373, and 375-379 to make them appear without any shading. In other embodiments the display generation engine 143 may additionally and/or alternatively modify the graphical representations of the non-selected organs by making them appear more “dim”, removing them from the electronic display 300, placing an “X” through them, and/or otherwise modifying them. In response to the user selection of graphical representations of the liver and the heart, the display generation engine 143 further removes the bar graphs 382a, 383a, and 385a-389a to demonstrate the corresponding organs are non-selected.

In response to the user selection of graphical representations of the liver and the heart, the display generation engine 143 further requests a new overall health risk score for the time period be calculated that takes into account only those test values and regression correlation coefficients for medical test results that correspond to the selected liver and heart. The overall HRS engine 141 may calculate the new overall health risk score by setting test values for medical test results that correspond to the non-selected organs to zero and using only the test values for the medical test results that correspond to the selected liver and heart. The display generation engine 143 modifies the overall health risk score bar graph 350 in FIG. 3C to reflect the newly calculated overall health risk score. This enables the user to select one or more desired organs and view the overall health risk score for those selected organs. When certain organs are selected the user may further adjust the time period adjustable user interface element 357 along the timeline 355 to select a desired earlier time period and cause the display generation engine 143 to update the overall HRS for the earlier time period, taking into account only those test values (for the earlier time period) and regression correlation coefficients for medical test results that correspond to the selected liver and heart. The organ health risk scores for the liver and the heart may also be updated for the earlier time period. A user may further “reselect” previously non-selected organs using user interface input device 104 to bring the test values for those organs back into the overall HRS calculation.

Referring again to FIG. 2, in some implementations therapy input engine 144 may receive therapy input from one or more of the user interface input device(s) 104, one or more of the biometric input device(s) 108, and/or other sources. The therapy input is indicative of actions performed and/or performable by a patient associated with the patient identifier and the therapy input engine 144 utilizes the therapy input to determine predicted changes to test values of a patient identifier that may result based on the therapy input. The therapy input engine 144 provides the predicted changes to the overall HRS engine 141 and/or the organ HRSs engine 142 to determine one or more adjusted overall HRSs and/or one or more adjusted organ HRSs based on those predicted changes. The display generation engine 143 may utilize the one or more adjusted overall HRSs and/or one or more adjusted organ HRSs in generating an electronic display, and may optionally display one or more aspects of the therapy input in the electronic display.

Examples are provided with reference to FIGS. 4A-4C. FIG. 4A illustrates another example of an electronic display 400 that may be generated by the display generation engine 143. The electronic display 400 includes an avatar 460 for a patient with an indication of biometric data therapy input 462, an indication of an overall health risk score for the patient 450, with graphical representations of organs 471-479 provided in anatomically appropriate positions in the avatar 460. The graphical representations of organs 471-479 include graphical representations of: a pancreas 471, a liver 472, kidneys 473, a heart 474, a gastrointestinal tract 475, a bone 476, a brain 477, a lung 478, and a prostate 479. In other implementations, more or fewer graphical representations of organs may be displayed.

The display generation engine 143 has determined display properties for each of the graphical representations of the organs 471-479 based on a magnitude of respective individual organ health risk scores for the organs at a time period of “today”—such as in a similar manner as that described above with respect to FIG. 3A. The electronic display 400 also includes additional graphical representations of organs (481-489) provided to the left of the avatar 460. The additional graphical representations of organs 481-489 include graphical representations of: a pancreas 481, a liver 482, kidneys 483, a heart 484, a gastrointestinal tract 485, a bone 486, a brain 487, a lung 488, and a prostate 489. In other implementations, more or fewer (e.g., none) additional graphical representations of organs may be displayed. The display generation engine 143 has determined display properties for each of the additional graphical representations of the organs 481-489 that match the display properties of the graphical representations of organs 471-479, such as in a similar manner as that described above with respect to FIG. 3A.

The electronic display 400 also includes additional display properties 481a-489a provided to the right of respective of the additional graphical representations of organs 481-489. Each of the additional display properties is a numerical indication that provides a scalar indication of the individual organ health risk score of a corresponding of the organs—and may optionally be provided with shading that matches the shading of a corresponding of the additional graphical representations of the organs 481-489. The display generation engine 143 has ordered the additional graphical representations of organs 481-489 and the additional display properties 481a-489a based on the respective individual organ health risk scores.

The electronic display 400 also includes an overall health risk score numerical indication 450 and bar graph 451 that provide visual indications of the overall health risk score for “today”. The electronic display 400 also includes a time period adjustable user interface element 457 that indicates the display properties of the electronic display 400 are being displayed for “today”. the time period adjustable user interface element 457 may be adjusted along the timeline 455 to the left to select a desired earlier time period, or to the right to select a desired future time period, and cause the display generation engine 143 to update the electronic display based on an overall HRS and organ HRSs for the selected time period.

In FIG. 4A an indication of biometric data therapy input 462 is provided that displays a measured heart rate value (92 beats per minute), calories intake/dietary value (4600 calories), BMI value (32.3), activity value (872 steps), and sleep value (6.5 hours) of the patient. The values may represent averages or other statistical measure over the last day, week, two weeks, or other time period. In some implementations the values may be measured by a biometric data input device 108 of the patient and/or user inputted in the biometric data input device 108 by the patient. As described herein, in some implementations, the therapy input engine 144 may utilize the displayed therapy input (and optionally additional therapy input) to adjust test values for a patient identifier of the patient based on therapy adjustment values database 158. For example, the therapy input engine 144 may utilize the displayed therapy input and additional therapy input received since the last medical tests for the patient were administered, to adjust test values of one or more of those last medical tests. The therapy input engine 144 may send the adjusted values to the overall HRS engine 141 and/or the organ HRSs engine 142 to determine one or more adjusted overall HRSs and/or one or more adjusted organ HRSs based on those predicted changes. The display generation engine 143 has utilized those adjusted values in generating the overall health risk score numerical indication 450 and bar graph 451, the graphical representations of the organs 471-479, 481-489, and/or the additional display properties 481a-489-a of FIG. 4A.

FIG. 4B illustrates the example electronic display 400 of FIG. 4A, with the electronic display 400 modified based on anticipated biometric data therapy input (illustrated in the indication of anticipated biometric data therapy input 462) and a user time period adjustment to a future time period. The modified electronic display 400 includes a modified indication 450/451 of an overall health risk score for the patient calculated based on the anticipated biometric data therapy input and a future time period and graphical representations of organs 471-489/481-489 with modified display properties of the organs determined based on organ health risk scores for the organs calculated based on the anticipated biometric data therapy input and the future time period.

In FIG. 4B an indication of anticipated biometric data therapy input 462 is provided that displays an anticipated heart rate value (81 beats per minute), an anticipated weight/dietary value (3575 calories), an anticipated BMI value (28.5), an anticipated activity value (4800 steps), and an anticipated sleep value (6.5 hours) of the patient. In FIG. 4B, the user has adjusted the time period adjustable user interface element 457 to a future time period. The display generation engine 143 receives an indication of the adjustment to the future time period and, in response to the adjustment, updates the electronic display 400 based on an overall HRS and organ HRSs calculated for the future time period in view of one or more aspects of the anticipated future biometric data therapy input 462.

For example, the therapy input engine 144 may utilize or more aspects of the anticipated future biometric data therapy input 462 to adjust test values of one or more of the last medical tests of the patient (or test values that have been adjusted in view of measured actual therapy input). The therapy input engine 144 may send the adjusted values to the overall HRS engine 141 and/or the organ HRSs engine 142 to determine one or more adjusted overall HRSs and/or one or more adjusted organ HRSs based on those predicted changes. The display generation engine 143 utilized those adjusted values in generating the overall health risk score numerical indication 450 and bar graph 451, the graphical representations of the organs 471-479, 481-489, and/or the additional display properties 481a-489-a of FIG. 4B. In particular, the electronic display 400 has been modified to provide an indication of an overall health risk score 450/451 for the patient at the future time period that indicates a lesser likelihood of increased need for medical care and costs than the overall health risk score 450 of FIG. 4A. The electronic display 400 has also been modified to update the numerical indicators of multiple of the additional display properties 481a-489a to reflect adjusted individual organ health risk scores for corresponding organs at the future time period.

In some implementations, the anticipated future biometric data therapy input may be based on previously measured actual biometric data therapy input and an assumption that the same or similar therapy will persist through to the future time period. In some implementations, a user may utilize one of the user interface input devices 104 to manually adjust the input to reflect target goals of the user (e.g., via interaction with 462). Accordingly, the user may provide therapy input goals via the user interface input devices 104 and visualize the impact the therapy input goals may have on an overall health risk score and/or individual organ health risk scores at one or more future time periods.

FIG. 4C illustrates the example electronic display of FIG. 4B, with the display modified to provide an illness map 465 that provides in depth analysis for a series of computations based on medical test results associated with calculation of illness stage/progression, illness severity, illness complexity, and test volatility over time. This additional detail refines and further specifies the overall health risk score of FIG. 4B. It also impacts the computation for the overall health risk score based on such factors as volatility of test results over time, and increasing influence of dysfunctional complexity in co-existing organs.

The user may cause the additional detail for the illness map 465 to be displayed based on selecting the illness map 465 in FIG. 4B, thereby causing it to “expand.” The illness map 465 in FIG. 4C volatility displays scores for the illness severity component, the illness volatility component, the illness complexity component, and the disease stage/progression component of the overall health risk score of FIG. 4B. As described herein, the overall HRS engine 141 may calculate the score for a given component based on applying the regression coefficients for the given component to the test values of the patient identifier for the medical test results associated with the regression coefficients for the given component. For example, to determine a score for the illness volatility component, the coefficient for illness volatility may be multiplied by a normalized test value that is determined based on fluctuation over time between the z-scores of medical test result(s) for serum creatinine and serum BUN for the patient identifier. The illness map 465 may be expanded for other time periods and/or based on other (or no) therapy inputs and the illness severity component, the illness volatility component, the illness complexity component, and/or the disease stage/progression component will reflect the components of the overall health risk score for those time periods and/or therapy inputs. Although the scores in the illness map 465 are illustrated in FIG. 4C as numerical values scaled from zero to five, in other implementations other scales may be utilized. Also, in some implementations a horizontal bar graph (e.g., similar to bar graph 451) may additionally and/or alternatively be displayed for each of the scores. For example, a horizontal bar graph may be provided for the progression component with approximately 60% of the bar graph shaded to illustrate a progression component score of three on a five point scale.

In some implementations, the electronic displays 300 and/or 400 may provide the user the option to provide input to display the z-scores and/or regression correlation coefficients utilized to calculate the organ health risk score associated with one or more of the organs and/or to calculate the overall health risk score and/or one or more components thereof. For example, a graphical representation of an organ may be long-clicked to provide the z-scores and/or regression correlation coefficients utilized to calculate the organ health risk score for the organ. In other implementations, the z-scores and/or regression correlation coefficients utilized to calculate the organ health risk score associated with one or more of the organs and/or to calculate the overall health risk score and/or one or more components thereof may be displayed in one or more of the electronic displays 300 and/or 400 without requiring explicit user input. Although particular graphical representations of organs are illustrated in the example electronic displays 300 and 400, additional and/or alternative graphical representations may be used such as, for example, an alternative bone instead of the femur, a heart shape instead of the anatomical heart, other shapes or symbols, etc.

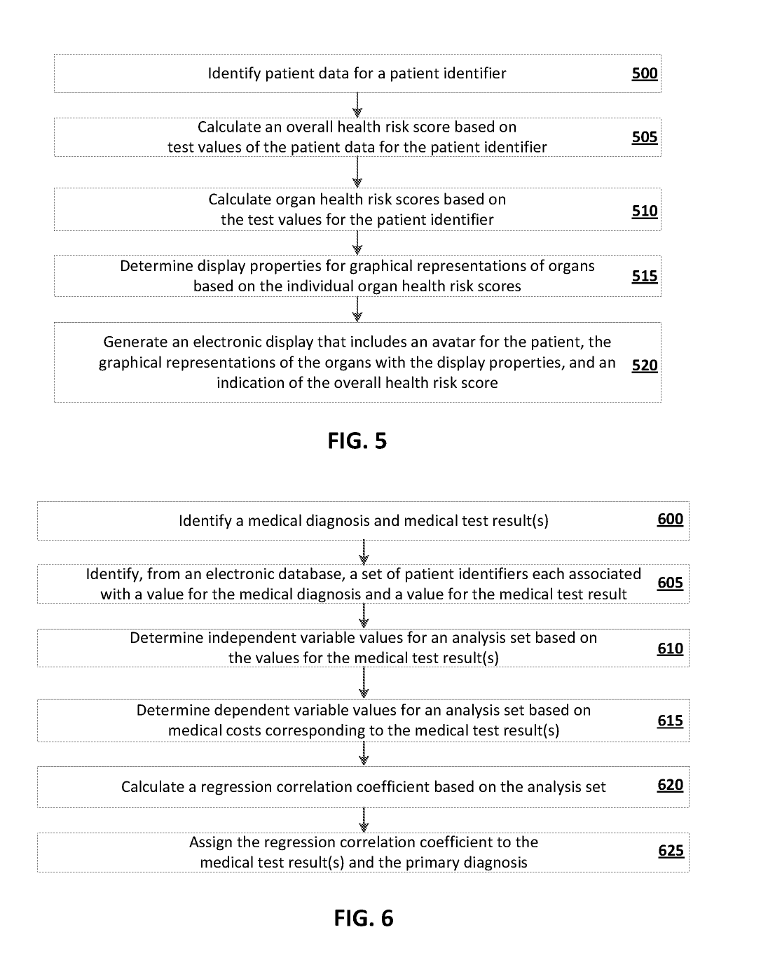

FIG. 5 is a flow chart illustrating an example method of calculating an overall health risk score and organ health risk scores based on patient data and generating an electronic display with display properties determined based on the calculated scores. Other implementations may perform the steps in a different order, omit certain steps, and/or perform different and/or additional steps than those illustrated in FIG. 5. For convenience, aspects of FIG. 5 will be described with reference to a system of one or more computers that perform the process. The system may include, for example, one or more of the engines 141-143 of patient presentation system 140.

At step 500, patient data for a patient identifier is identified. For example, the system may identify patient data from patient data database 154 and/or from one or more other sources. The patient data defines a medical diagnosis for the patient identifier and test values for medical test results of the patient identifier. For example, the patient data may identify an ICD value that indicates the patient has CKD.

At step 505, an overall health risk score is calculated based on test values of the patient data for the patient identifier. For example, the system may use the test values for the patient identifier and regression correlation coefficients for the medical diagnosis (e.g., from regression correlation coefficients database 156) to calculate an overall health risk score for the patient identifier. In some implementations, the system uses the regression coefficients for the medical diagnosis and the test values for the patient identifier to calculate an overall health risk score for the patient identifier for each of one or more time periods. For example, the system may apply test values for medical test results of a first time period to the regression correlation coefficients to determine a first overall health risk score for the first time period, may apply test values for medical test results of a second time period to the regression correlation coefficients to determine a second overall health risk score for the second time period, etc.

At step 510, organ health risk scores are calculated based on the test values for the patient identifier. For example, the system may calculate an organ health risk score for an organ of the patient identifier based on applying one or more selected test values for one or more medical test results (for the organ) to the regression correlation coefficients for those one or more medical test results (for the organ). In some implementations, the system determines organ health risk scores for each of multiple time periods. For example, the system may determine, for a first time period, a first organ risk score for the heart, a first organ risk score for the liver, a first organ risk score for the pancreas, etc.; determine, for a second time period, a second organ risk score for the heart, a second organ risk score for the liver, a second organ risk score for the pancreas, etc.; and so forth.

At step 515, display properties are determined for graphical representations of organs based on the individual organ health risk scores. For example, the system may determine display properties for one or more of the organs for a given time period that are particularized to one or more of the organ health risk scores for the time period. For example, a display property of a graphical representation of a pancreas may be a color of “red” based on an organ health risk score for the pancreas indicating a large degree of dysfunction of the organ; whereas a display property of a graphical representation of a heart may be a color of “yellow” based on a health risk score for the heart indicating a mild degree of dysfunction of the organ. In some implementations, the system may also determine display properties for an indication of the overall health risk score based on the calculated overall health risk score.

At step 520, an electronic display is generated that includes an avatar for the patient, the graphical representations of the organs with the display properties, and an indication of the overall health risk score. The system may provide the electronic display to one or more users via one or more user interface output devices. In implementations in which overall health risk score and organ risk scores are determined for the patient identifier for each of a plurality of time periods, the system may determine (at step 515) display properties for each of the time periods and at step 520 may update the electronic display to show the display properties for each of those time periods (automatically or in response to user input).

FIG. 7 is a block diagram of an example computer system 710. Computer system 710 typically includes at least one processor 714 which communicates with a number of peripheral devices via bus subsystem 712. These peripheral devices may include a storage subsystem 724, including, for example, a memory subsystem 725 and a file storage subsystem 726, user interface input devices 722, user interface output devices 720, and a network interface subsystem 716. The input and output devices allow user interaction with computer system 710. Network interface subsystem 716 provides an interface to outside networks and is coupled to corresponding interface devices in other computer systems.

User interface input devices 722 may include a keyboard, pointing devices such as a mouse, trackball, touchpad, or graphics tablet, a scanner, a touchscreen incorporated into the display, audio input devices such as voice recognition systems, microphones, and/or other types of input devices. In general, use of the term “input device” is intended to include all possible types of devices and ways to input information into computer system 710 or onto a communication network.

User interface output devices 720 may include a display subsystem, a printer, a fax machine, or non-visual displays such as audio output devices. The display subsystem may include a cathode ray tube (CRT), a flat-panel device such as a liquid crystal display (LCD), a projection device, including holographic devices or some other mechanism for creating a visible image. The display subsystem may also provide non-visual display such as via audio output devices. In general, use of the term “output device” is intended to include all possible types of devices and ways to output information from computer system 710 to the user or to another machine or computer system.

Storage subsystem 724 stores programming and data constructs that provide the functionality of some or all of the modules described herein. For example, the storage subsystem 724 may include the logic to perform one or more of the methods described herein such as, for example, the methods of FIGS. 5 and/or 6.

These software modules are generally executed by processor 714 alone or in combination with other processors. Memory 725 used in the storage subsystem can include a number of memories including a main random access memory (RAM) 730 for storage of instructions and data during program execution and a read only memory (ROM) 732 in which fixed instructions are stored. A file storage subsystem 726 can provide persistent storage for program and data files, and may include a hard disk drive, a floppy disk drive along with associated removable media, a CD-ROM drive, an optical drive, or removable media cartridges. The modules implementing the functionality of certain implementations may be stored by file storage subsystem 726 in the storage subsystem 724, or in other machines accessible by the processor(s) 714.

Bus subsystem 712 provides a mechanism for letting the various components and subsystems of computer system 710 communicate with each other as intended. Although bus subsystem 712 is shown schematically as a single bus, alternative implementations of the bus subsystem may use multiple busses.

Computer system 710 can be of varying types including a workstation, server, computing cluster, blade server, server farm, or any other data processing system or computing device. Due to the ever-changing nature of computers and networks, the description of computer system 710 depicted in FIG. 7 is intended only as a specific example for purposes of illustrating some implementations. Many other configurations of computer system 710 are possible having more or fewer components than the computer system depicted in FIG. 7.

While several implementations have been described and illustrated herein, a variety of other means and/or structures for performing the function and/or obtaining the results and/or one or more of the advantages described herein may be utilized, and each of such variations and/or modifications is deemed to be within the scope of the implementations described herein. More generally, all parameters, dimensions, materials, and configurations described herein are meant to be exemplary and that the actual parameters, dimensions, materials, and/or configurations will depend upon the specific application or applications for which the teachings is/are used. Those skilled in the art will recognize, or be able to ascertain using no more than routine experimentation, many equivalents to the specific implementations described herein. It is, therefore, to be understood that the foregoing implementations are presented by way of example only and that, within the scope of the appended claims and equivalents thereto, implementations may be practiced otherwise than as specifically described and claimed. Implementations of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

1. A computer-implemented method, comprising:
identifying, from one or more electronic databases, patient data for a patient identifier, the patient data comprising test values based on results for one or more selected medical tests for a patient associated with the patient identifier;
calculating, utilizing one or more processors, an overall health risk score for the patient identifier based on the test values;
calculating, utilizing one or more of the processors, organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
generating an electronic display that includes an indication of the overall health risk score and indications of the organ health risk scores;
identifying therapy input indicative of actions performed or performable by the patient, the therapy input provided from a separate user interface input device or one or more separate biometric input devices;
calculating, utilizing one or more of the processors, a second overall health risk score for the patient identifier based on the test values and the therapy input;
calculating, utilizing one or more of the processors, second organ health risk scores for the patient identifier based on the test values and the therapy input; and
generating a modified electronic display that includes an indication of the second overall health risk score and indications of the second organ health risk scores.

2. A computer-implemented method, comprising:
identifying, from one or more electronic databases, patient data for a patient identifier, the patient data comprising test values based on results for one or more selected medical tests for a patient associated with the patient identifier;
calculating, utilizing one or more processors, an overall health risk score for the patient identifier based on the test values;
calculating, utilizing one or more of the processors, organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
determining display properties for graphical representations of the organs based on the organ health risk scores, wherein each of the display properties is determined for a corresponding of the organs, and wherein a given display property for the given organ is determined based on a magnitude of the given organ health risk score; and
generating an electronic display that includes an avatar for the patient, the graphical representations of the organs with the display properties, and an indication of the overall health risk score;
wherein the electronic display includes the graphical representations of the organs with the display properties in anatomically appropriate positions in the avatar.

3. The computer implemented method of claim 2, wherein the test values include a first group of test values associated with a first time period and a second group of test values associated with a second time period, and wherein the overall health risk score and the organ health risk scores are calculated based on the first group of test values, and further comprising:
calculating, utilizing one or more of the processors, a second overall health risk score for the patient identifier based on the second group of test values;
calculating, utilizing one or more of the processors, second organ health risk scores for the patient identifier based on the second group of test values, wherein a given second organ health risk score of the second organ health risk scores for the given organ is calculated based on one or more of the second group of test values that are indicative of function of the given organ;
determining second display properties for the graphical representations of the organs based on the second organ health risk scores; and
modifying the electronic display to display the graphical representations of the organs with the second display properties, the second overall health risk score, and an indication of the second time period.

4. The method of claim 3, further comprising:
receiving a time period adjustment input;
wherein modifying the electronic display to display the graphical representations of the organs with the second display properties, the second overall health risk score, and the indication of the second time period is in response to receiving the time period adjustment input.

5. The method of claim 4, wherein the time period adjustment input is received responsive to user interaction with an adjustable user interface element of the electronic display and wherein the indication of the second time period is based on a current state of the adjustable user interface element.

6. The method of claim 2, further comprising:
determining additional display properties associated with additional graphical representations of multiple of the organs, wherein each of the additional display properties is determined based on one of the organ health risk scores and is determined for a corresponding of the organs, and wherein a given additional display property associated with the given organ is determined based on a magnitude of the given organ health risk score and provides more detailed information than the given display property for the given organ;
wherein generating the electronic display further includes generating the additional graphical representations of the multiple of the organs along with the additional display properties, the additional graphical representations and the additional display properties depicted exterior of the avatar in the electronic display.

7. The method of claim 6, further comprising ordering the additional graphical representations of the multiple of the organs in the electronic display based on the organ health risk scores.

8. The method of claim 7, wherein the given additional display property comprises at least one of:
a numerical indication of the given organ health risk score; and
a bar graph indicating a magnitude of the organ health risk score.

9. The method of claim 2, wherein the display properties include a plurality of colors each mapped to one or more of the organ health risk scores.

10. The method of claim 2, further comprising:
identifying therapy input indicative of actions performed or performable by the patient;
calculating, utilizing one or more of the processors, a modified overall health risk score for the patient identifier based on the test values and the therapy input; and
modifying the electronic display to display the modified overall health risk score.

11. The method of claim 10, wherein the therapy input is received from a personal fitness monitoring device of the patient and indicates actions performed by the patient.

12. The method of claim 10, wherein the therapy input includes actions performable by the patient to improve the overall health risk score and the modified overall health risk score indicates an anticipated potential future health risk score if the actions performable by the patient are actually performed by the patient.

13. The method of claim 10, further comprising:
calculating, utilizing one or more of the processors, anticipated future organ health risk scores for the patient identifier based on the test values and the therapy input;
determining second display properties for the graphical representations of the organs based on the anticipated future organ health risk scores; and
modifying the electronic display to display the graphical representations of the organs with the second display properties along with the modified overall health risk score.

14. The method of claim 2, wherein calculating the overall health risk score for the patient identifier based on the test values comprises:
identifying coefficient values for each of the test values, the coefficient values indicating a statistically calculated historical impact, for a medical diagnosis of the patient, of the test values on predicting an increased need for medical care and associated costs; and
modifying each of the test values in view of a respective of the coefficient values.

15. The method of claim 14, wherein the test values are z-scores.

16. The method of claim 2, wherein calculating the overall health risk score for the patient identifier based on the test values comprises one or more of:
calculating an illness severity component of the overall health risk score based on a ratio of Blood Urea Nitrogen levels and serum Creatinine levels of the patient as determined based on one or more of the test values; and
calculating an illness volatility component of the overall health risk score based on variations over time for the Blood Urea Nitrogen levels of the patient and variations over time for the serum Creatinine levels of the patient, as determined based on one or more of the test values.

17. The method of claim 16, further comprising:
determining an illness severity graphical indicator based on the magnitude of the illness severity component of the overall health risk score;
determining an illness complexity graphical indicator based on the magnitude of the illness complexity component of the overall health risk score; and
including the illness severity graphical indicator and the illness complexity graphical indicator in the electronic display.

18. The method of claim 16, further comprising:
receiving a selection for additional information related to the overall health risk score;
wherein including the illness severity graphical indicator and the illness complexity graphical indicator in the electronic display comprises modifying the display in response to receiving the selection.

19. The method of claim 2, wherein calculating the overall health risk score for the patient identifier based on the test values comprises one or more of:
calculating a disease stage/progression component of the overall health risk score based on disease progression data of the patient data, the disease progression data indicating an extent of a medical diagnosis of the patient; and
calculating an illness complexity component of the overall health risk score, the illness complexity component based on one or more selected test values of the test values, the selected test values excluding medical test results that define the medical diagnosis of the patient.

20. The method of claim 2, wherein the selected medical tests include at least one physical measurement medical test and at least one laboratory measurement medical test.

21. The method of claim 2, wherein the test values include values based on one or more of: complete blood counts with white cell differential, serum electrolytes, liver profile enzymes, metabolic study values, estimated glomerular filtration rate, urine analysis, selected tumor markers, genetic markers, thyroid, lipid and tri-glyceride values, total cholesterol and ratio high-density lipoprotein (HDL) & low-density lipoprotein (LDL), blood pressure, body mass index, waist circumference, and/or patient health questionnaire-9 (PHQ-9) results.

22. A system, comprising:
one or more processors;
memory storing instructions, the instructions comprising instructions that, when executed by the one or more processors, cause the processors to:
identify patient data for a patient identifier, the patient data comprising test values based on results for one or more selected medical tests for a patient associated with the patient identifier;
calculate an overall health risk score for the patient identifier based on the test values;
calculate organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
determine display properties for graphical representations of the organs based on the organ health risk scores, wherein each of the display properties is determined for a corresponding of the organs, and wherein a given display property for the given organ is determined based on a magnitude of the given organ health risk score; and
generate an electronic display that includes an avatar for the patient, the graphical representations of the organs with the display properties, and an indication of the overall health risk score;
wherein the electronic display includes the graphical representations of the organs with the display properties in anatomically appropriate positions in the avatar.

23. At least one non-transitory computer-readable medium comprising instructions that, in response to execution of the instructions by a computing system, cause the computing system to perform the following operations:
identify patient data for a patient identifier, the patient data comprising test values based on results for one or more selected medical tests for a patient associated with the patient identifier;
calculate an overall health risk score for the patient identifier based on the test values;
calculate organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
determine display properties for graphical representations of the organs based on the organ health risk scores, wherein each of the display properties is determined for a corresponding of the organs, and wherein a given display property for the given organ is determined based on a magnitude of the given organ health risk score; and
generate an electronic display that includes an avatar for the patient, the graphical representations of the organs with the display properties, and an indication of the overall health risk score;
wherein the electronic display includes the graphical representations of the organs with the display properties in anatomically appropriate positions in the avatar.

24. A computer-implemented method, comprising:
identifying, from one or more electronic databases, patient data for a patient identifier, the patient data comprising test values based on results for one or more selected medical tests for a patient associated with the patient identifier;
calculating, utilizing one or more processors, an overall health risk score for the patient identifier based on the test values;
calculating, utilizing one or more of the processors, organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
generating an electronic display that includes an indication of the overall health risk score and indications of the organ health risk scores;
identifying therapy input indicative of actions performed or performable by the patient;
calculating, utilizing one or more of the processors, a second overall health risk score for the patient identifier based on the test values and the therapy input;
calculating, utilizing one or more of the processors, second organ health risk scores for the patient identifier based on the test values and the therapy input; and
generating a modified electronic display that includes an indication of the second overall health risk score and indications of the second organ health risk scores.

25. The method of claim 24, wherein the therapy input is received from a personal fitness monitoring device of the patient and indicates actions performed by the patient.

26. The method of claim 24, further comprising:
determining display properties for graphical representations of the organs based on the organ health risk scores, wherein each of the display properties is determined for a corresponding of the organs, and wherein a given display property for the given organ is determined based on a magnitude of the given organ health risk score;
wherein the indications of the organ health risk scores in the electronic display comprise the graphical representations of the organs with the display properties.

27. The method of claim 26, further comprising:
determining second display properties for the graphical representations of the organs based on the second organ health risk scores, wherein each of the second display properties is determined for a corresponding of the organs; and
wherein the indications of the organ health risk scores in the modified electronic display comprise the graphical representations of the organs with the second display properties.

28. The method of claim 27, wherein the graphical representations of the organs in the electronic display are provided in anatomically appropriate positions in an avatar for the patient and wherein the graphical representations of the organs in the modified electronic display are provided in the anatomically appropriate positions in the avatar for the patient.

29. The method of claim 28, wherein the display properties include a first set of colors mapped to the organ health risk scores and the second display properties include a second set of colors mapped to the second organ health risk scores.

30. The method of claim 28, further comprising:
determining additional display properties associated with additional graphical representations of multiple of the organs, wherein each of the additional display properties is determined based on the organ health risk scores and is determined for a corresponding of the organs, and wherein a given additional display property associated with the given organ is determined based on a magnitude of the given organ health risk score and provides more detailed information than the given display property for the given organ;
wherein generating the electronic display further includes generating the additional graphical representations of the multiple of the organs along with the additional display properties, the additional graphical representations and the additional display properties depicted exterior of the avatar in the electronic display;
determining second additional display properties associated with second additional graphical representations of multiple of the organs, wherein each of the second additional display properties is determined based on the second organ health risk scores and is determined for a corresponding of the organs;
wherein generating the modified electronic display further includes generating the second additional graphical representations of the multiple of the organs along with the additional second display properties, the additional second graphical representations and the additional second display properties depicted exterior of the avatar in the modified electronic display.

31. The method of claim 30, further comprising:
ordering the additional graphical representations of the multiple of the organs in the electronic display based on the organ health risk scores; and
ordering the additional second graphical representations of the multiple of the organs in the modified electronic display based on the second organ health risk scores.

32. The method of claim 24, wherein calculating the second overall health risk score for the patient identifier based on the test values and the therapy input comprises:
identifying, based on the therapy input, a predicted change for each of one or more affected test values of the test values;
modifying each of the affected test values to create one or more modified test values, the modifying of a given affected test value of the affected test values comprising modifying the given affected test value in view of the predicted change for the given affected test value; and
calculating the modified overall health risk score based on the one or more modified test values.

33. The method of claim 24, wherein calculating the second organ health risk scores for the patient identifier based on the test values and the therapy input comprises:
identifying, based on the therapy input, a predicted change to a given test value of the one or more test values that are indicative of function of the given organ;
modifying the given test value based on the predicted change to create a modified given test value; and
calculating, for the given organ, a second organ health risk score of the second organ health risk scores based on the modified given test value.

34. The method of claim 33, wherein calculating the second organ health risk score based on the modified given test value comprises:
identifying a coefficient value for the modified given test value, the coefficient value indicating a statistically calculated historical impact, for a medical diagnosis of the patient, of the modified given test value on predicting an increased need for medical care and associated costs; and
modifying the modified given test value in view of the coefficient value.

35. The method of claim 24, wherein the therapy input includes actions performable by the patient to improve the overall health risk score and the second overall health risk score indicates an anticipated potential future health risk score if the actions performable by the patient are actually performed by the patient.

36. The method of claim 35, wherein the therapy input is received via user interaction with the electronic display.

37. The method of claim 24, wherein calculating the overall health risk score for the patient identifier based on the test values comprises one or more of:
calculating an illness severity component of the overall health risk score based on a ratio of Blood Urea Nitrogen levels and serum Creatinine levels of the patient as determined based on one or more of the test values; and
calculating an illness volatility component of the overall health risk score based on variations over time for the Blood Urea Nitrogen levels of the patient and variations over time for the serum Creatinine levels of the patient, as determined based on one or more of the test values.

38. The method of claim 37, further comprising:
determining an illness severity graphical indicator based on the magnitude of the illness severity component of the overall health risk score;
determining an illness complexity graphical indicator based on the magnitude of the illness complexity component of the overall health risk score; and
including the illness severity graphical indicator and the illness complexity graphical indicator in the electronic display.

39. The method of claim 38, further comprising:
receiving a selection for additional information related to the overall health risk score;
wherein including the illness severity graphical indicator and the illness complexity graphical indicator in the electronic display comprises modifying the display in response to receiving the selection.

40. A system, comprising:
one or more processors;
memory storing instructions, the instructions comprising instructions that, when executed by the one or more processors, cause the processors to:
identify, from one or more electronic databases, patient data for a patient identifier, the patient data comprising test values based on results for one or more selected medical tests for a patient associated with the patient identifier;
calculate an overall health risk score for the patient identifier based on the test values;
calculate organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
generate an electronic display that includes an indication of the overall health risk score and indications of the organ health risk scores;
identify therapy input indicative of actions performed or performable by the patient;
calculate a second overall health risk score for the patient identifier based on the test values and the therapy input;
calculate second organ health risk scores for the patient identifier based on the test values and the therapy input; and
generate a modified electronic display that includes an indication of the second overall health risk score and indications of the second organ health risk scores.

41. At least one non-transitory computer-readable medium comprising instructions that, in response to execution of the instructions by a computing system, cause the computing system to perform the following operations:
identify, from one or more electronic databases, patient data for a patient identifier, the patient data comprising test values based on results for one or more selected medical tests for a patient associated with the patient identifier;
calculate an overall health risk score for the patient identifier based on the test values;
calculate organ health risk scores for the patient identifier based on the test values, wherein each of the organ health risk scores is calculated for a corresponding organ of organs of the patient, and wherein a given organ health risk score of the organ health risk scores for a given organ of the organs is calculated based on one or more of the test values that are indicative of function of the given organ;
generate an electronic display that includes an indication of the overall health risk score and indications of the organ health risk scores;
identify therapy input indicative of actions performed or performable by the patient;
calculate a second overall health risk score for the patient identifier based on the test values and the therapy input;
calculate second organ health risk scores for the patient identifier based on the test values and the therapy input; and
generate a modified electronic display that includes an indication of the second overall health risk score and indications of the second organ health risk scores.
